(19) United States
(12) Patent Application Publication
(10) Pub. No.: US 2009/0118213 A1
(43) Pub. Date: May 7, 2009

(54) RNA ANTAGONIST COMPOUNDS FOR THE INHIBITION OF APO-B100 EXPRESSION

(76) Inventors: Henrik Frydenlund Hansen, Rodovre (DK); Bo Hansen, Hellerup (DK); Majken Westergaard, Birkerod (DK); Ellen Marie Straarup, Birkerod (DK); Christoph Rosenbohm, Birkerod (DK)

Correspondence Address: FISH & RICHARDSON PC, P.O. BOX 1022, MINNEAPOLIS, MN, 55440-1022, US

(21) Appl. No.: 12/066,852

(22) PCT Filed: Sep. 1, 2006
(86) PCT No.: PCT/DK06/00481

§ 371 Date: Jun. 13, 2008

Related U.S. Application Data
(60) Provisional application No. 60/718,018, filed on Sep. 15, 2005.
(60) Provisional application No. 60/796,211, filed on Apr. 27, 2006.

(30) Foreign Application Priority Data
Apr. 27, 2006 (DK) PA 200600598

(51) Int. Cl. A61K 31/7088; C07H 21/04

(52) U.S. Cl. 514/44; 536/22.1

(57) ABSTRACT

Oligonucleotides directed against the Apo-B100 gene are provided for modulating the expression of Apo-B100. The compositions comprise oligonucleotides, particularly antisense oligonucleotides, targeted to nucleic acids encoding the Apo-B100. Methods of using these compounds for modulation of Apo-B100 expression and for the treatment of diseases associated with either overexpression of Apo-B100, expression of mutated Apo-B100 or both are provided. Examples of diseases are cancer such as lung, breast, colon, prostate, pancreas, lung, liver, thyroid, kidney, brain, testes, stomach, intestine, bowel, spinal cord, sinuses, bladder, urinary tract or ovaries cancers. The oligonucleotides may be composed of deoxyribonucleosides or a nucleic acid analogue such as for example locked nucleic acid or a combination thereof.

### FIELD OF THE INVENTION

The present invention provides compositions and methods for modulating the expression of Apo-B100. In particular, this invention relates to oligonucleotide compounds which specifically hybridise to with nucleic acids encoding Apo-B100. The oligonucleotide compounds have been shown to modulate the expression of Apo-B100 and pharmaceutical preparations thereof and their use as treatment of cancer diseases are disclosed.

### BACKGROUND OF THE INVENTION

Apolipoprotein B (also known as ApoB, apolipoprotein B-100; ApoB-100, apolipoprotein B-48; ApoB-48 and Ag(x) antigen), is a large glycoprotein that serves an indispensable role in the assembly and secretion of lipids and in the transport and receptor-mediated uptake and delivery of distinct classes of lipoproteins. ApoB plays an important role in the regulation of circulating lipoprotein levels, and is therefore relevant in terms of atherosclerosis susceptibility which is highly correlated with the ambient concentration of apolipoprotein B-containing lipoproteins. See Davidson and Shelness (Annul Rev. Nutr., 2000, 20, 169-193) for further details of the two forms of ApoB present in mammals, their structure and medicinal importance of ApoB.

Elevated plasma levels of the ApoB-100-containing lipoprotein Lp(a) are associated with increased risk for atherosclerosis and its manifestations, which may include hypercholesterolemia (Seed et al., N. Engl. J. Med., 1990, 322, 1494-1499), myocardial infarction (Sandkamp et al., Clin. Chew., 1990, 36, 20-23), and thrombosis (Nowak-Gottl et al., Pediatrics, 1997, 99, Eli).

The plasma concentration of Lp(a) is strongly influenced by heritable factors and is refractory to most drug and dietary manipulation (Katan and Beynen, Am. J. Epidemiol., 1987, 125, 387-399; Vessby et al., Atherosclerosis, 1982, 44, 61-71). Pharmacologic therapy of elevated Lp(a) levels has been only modestly successful and apheresis remains the most effective therapeutic modality (Hajjar and Nachman, Annul Rev. Med., 1996, 47, 423-442).

Two forms of apolipoprotein B exist in mammals. ApoB-100 represents the full-length protein containing 4536 amino acid residues synthesized exclusively in the human liver (Davidson and Shelness, Annul Rev. Nutr., 2000, 20, 169-193). A truncated form known as ApoB-48 is collinear with the amino terminal 2152 residues and is synthesized in the small intestine of all mammals (Davidson and Shelness, Annul Rev. Nutr., 2000, 20, 169-193).

The basis by which the common structural gene for apolipoprotein B produces two distinct protein isoforms is a process known as RNA editing. A site specific cytosine-to-uracil editing reaction produces a UAA stop codon and translational termination of apolipoprotein B to produce ApoB-48 (Davidson and Shelness, Annul Rev. Nutr., 2000, 20, 169-193).

The medicinal significance of mammalian ApopB has been verified using transgenic mice studies either over expressing human ApoB (Kim and Young, J. Lipid Res., 1998, 39, 703-723; Nishina et al., J. Lipid Res., 1990, 31, 859-869) or ApoB knock-out mice (Farese et al., Proc. Natl. Acad. Sci. U.S.A., 1995, 92, 1774-1778; Kim and Young, J. Lipid Res., 1998, 39, 703-723).

To date, strategies aimed at inhibiting apolipoprotein B function have been limited to Lp(a) apheresis, antibodies, antibody fragments and ribozymes. Moreover, low biostability and/or low binding affinity antisense oligonucleotides have been disclosed and claimed in PCT publication WO 00/97662, WO 03/11887 and WO 2004/44181.

Consequently, there remains a need for additional agents capable of effectively antagonize apolipoprotein B function and consequently lower the plasma Lp(a) level.

The present invention provides effective Locked Nucleic Acid (LNA) oligomeric compounds and their use in methods for modulating apolipoprotein B expression, including inhibition of the alternative isoform of apolipoprotein B ApoB-48.

### SUMMARY OF THE INVENTION

The present invention provides compositions and methods for modulating the expression of apolipoprotein B (Apo-B100/Apo-B48). In particular, this invention relates to oligonucleotide compounds over specific motifs targeting apolipoprotein B. These motifs are SEQ ID NOS: 2-26, in particular SEQ ID NOS: 2, 3, 10, 11 and 21. Specific designs of LNA containing oligonucleotide compounds are also disclosed. Specifically preferred compounds are SEQ ID NOS: 29-47, in particular SEQ ID NOS: 29, 30, 31, 36, 37, 38, 40 and 42. The compounds of the invention are potent inhibitors of apoliprotein mRNA and protein expression. In vitro SEQ ID NOS: 29 and 30 down-regulated ApoB expression with IC₅₀ around 1-5 nM, and SEQ ID No 37 showed an IC₅₀ of about 0.5 nM. In vivo, the ApoB-100 mRNA expression was suppressed in the liver and jejunum following treatment with SEQ ID NO: 29 in a dose dependent manner. Concomitant with reduced ApoB-100 levels, the total cholesterol in plasma was lowered by 70%.

Pharmaceutical and other compositions comprising the oligonucleotide compounds of the invention are also provided. Further provided are methods of modulating the expression of apolipoprotein B. in cells or tissues comprising contacting said cells or tissues with one or more of the oligonucleotide compounds or compositions of the invention. Also disclosed are methods of treating an animal or a human, suspected of having or being prone to a disease or condition, associated with expression of apolipoprotein B by administering a therapeutically or prophylactically effective amount of one or more of the oligonucleotide compounds or compositions of the invention. Further, methods of using oligonucleotide compounds for the inhibition of expression of apolipoprotein B and for treatment of diseases associated with apolipoprotein B activity are provided. Examples of such diseases are different types of HDL/LDL cholesterol imbalance; dyslipidemias, e.g., familial combined hyperlipidemia (FCHL), acquired hyperlipidemia, hypercholestorolemia; statin-resistant hypercholesterolemia; coronary artery disease (CAD) coronary heart disease (CHD) atherosclerosis.

### BRIEF DESCRIPTION OF THE FIGURES

FIG. 1A: Relative ApoB mRNA expression in mouse hepatocytes (Hepa1-6 cells) following lipid-assisted uptake of SEQ ID NO: 29, siRNA (unmodified) or cholesteryl modified siRNA.

FIG. 1B: Relative ApoB expression in BNLCL2 cells following treatment with SEQ ID NOS: 29 and 30. Both compounds are potent inhibitors of the ApoB-100 mRNA already at 1 nM or 5 nM concentration.

FIG. 2A: Relative ApoB-100 mRNA expression following treatment (daily dosing i.v for three days) with SEQ ID NO: 29, siRNA (unmodified) (SEQ ID NOs: 48/49) or cholesteryl modified siRNA (SEQ ID NOs: 50/49) in livers.

FIG. 2B: Relative ApoB-100 mRNA expression following treatment (daily dosing i.v for three days) with SEQ ID NO: 29, siRNA (unmodified) (SEQ ID NOs: 48/49) or cholesteryl modified siRNA (SEQ ID NOs: 50/49) in jejunum.

FIG. 3: Relative levels of cholesterol in plasma of mice treated with SEQ ID NO: 29, siRNA (unmodified) (SEQ ID NOs: 48/49) or cholesteryl modified siRNA (SEQ ID NOs: 50/49).

FIG. 4: In vitro ApoB-100 target downregulation in BNCL or Hepa 1-6 cells. Dose response effect of SEQ ID No: 29 and 37 on the ApoB mRNA level (normalised to GapDH) from in mouse cell lines.

FIG. 5A: In vivo ApoB-100 silencing in liver following LNA antisense treatment of C57BL/6 mice. The LNA antisense molecules were dosed one dose (6.25, 12.5 or 25 mg/kg) and the siRNA (50 mg/kg) 3 consecutive days in C57BL/6 mice. ApoB-100 expression was measured by qPCR and normalised to Gapdh. Data represent mean±SD (n=7).

FIG. 5B: In vivo ApoB-100 silencing in jejunum following LNA antisense treatment of C57BL/6 mice. The LNA antisense molecules were dosed one dose (6.25, 12.5 or 25 mg/kg) and the siRNA (50 mg/kg) 3 consecutive days in C57BL/6 mice. ApoB-100 expression was measured by qPCR and normalised to Gapdh. Data represent mean±SD (n=7).

FIG. 6A: Plasma cholesterol levels following LNA antisense treatment. The LNA antisense molecules were dosed one dose (6.25, 12.5 or 25 mg/kg) and the siRNA (50 mg/kg)3 consecutive days in C57BL/6 mice. LDL-cholesterol levels were determined using a colorimetric kit. Data represent mean±SD (n=7).

FIG. 6B: Plasma cholesterol levels following LNA antisense treatment. The LNA antisense molecules were dosed one dose (6.25, 12.5 or 25 mg/kg) and the siRNA (50 mg/kg)3 consecutive days in C57BL/6 mice. Plasma Total cholesterol levels were determined using a colorimetric kit. Data represent mean±SD (n=7).

FIG. 7: Shows the sequence comparison of the reverse compliment of the preferred sequences of the ApoB target nucleic acid, which have been used to design oligomeric compounds according to the invention.

FIG. 8: In vitro screen and dose response (1, 5 or 25 nM) in Huh-7 (Hepatocytes) cells treated with different LNA antisense oligonucleotides and the effect of the oligonucleotides measured as target mRNA (ApoB-100) down regulation (QPCR).

FIG. 9: IC50 (the concentration of antisense oligonucleotide to get 50% inhibition of target (ApoB-100) expression) for 7 selected LNA antisense oligonucleotides, measured in Huh-7 cells analysed by QPCR.

FIG. 10A: ApoB-100 mRNA levels measured in liver at sacrifice day 28. C57BL/6 mice were dosed either twice weekly with 2.5 mg/kg/dose (total of 8 doses) or once weekly 5 mg/kg (total of 4 doses) for 4 weeks.

FIG. 10B: Plasma LDL levels measured once weekly for 4 weeks in retro orbital blood. C57BL/6 mice were dosed either twice weekly with 2.5 mg/kg/dose (total of 8 doses) or once weekly 5 mg/kg (total of 4 doses) for 4 weeks.

FIG. 11A: Duration of action measured as ApoB-100 mRNA levels in liver at sacrifice at day 3, 5, 8, 13, or 21. C57BL/6 mice were dosed one, two or three doses of 25 mg/kg/dose SEQ ID NO:37 one dose at each of 1, 2 or 3 consecutive days, respectively.

FIG. 11B: Total plasma cholesterol measured at sacrifice day 3, 5, 8, 13 or 21. C57BL/6 female mice were dosed one, two or 3 doses of 25 mg/kg/dose of SEQ ID No: 37, one dose each day on one, two or three consecutive days, respectively.

### DETAILED DESCRIPTION OF THE INVENTION

The present invention employs oligomeric compounds, particularly antisense oligonucleotides, for use in modulating the function of nucleic acid molecules encoding apolipoprotein B (such as Apo-B100 and/or ApoB-48). The modulation is ultimately a change in the amount of apolipoprotein B produced. In one embodiment this is accomplished by providing oligomeric compounds, which specifically hybridise with nucleic acids, such as messenger RNA, which encodes apolipoprotein B. The modulation preferably results in the inhibition of the expression of apolipoprotein B, i.e. leads to a decrease in the number of functional proteins produced.

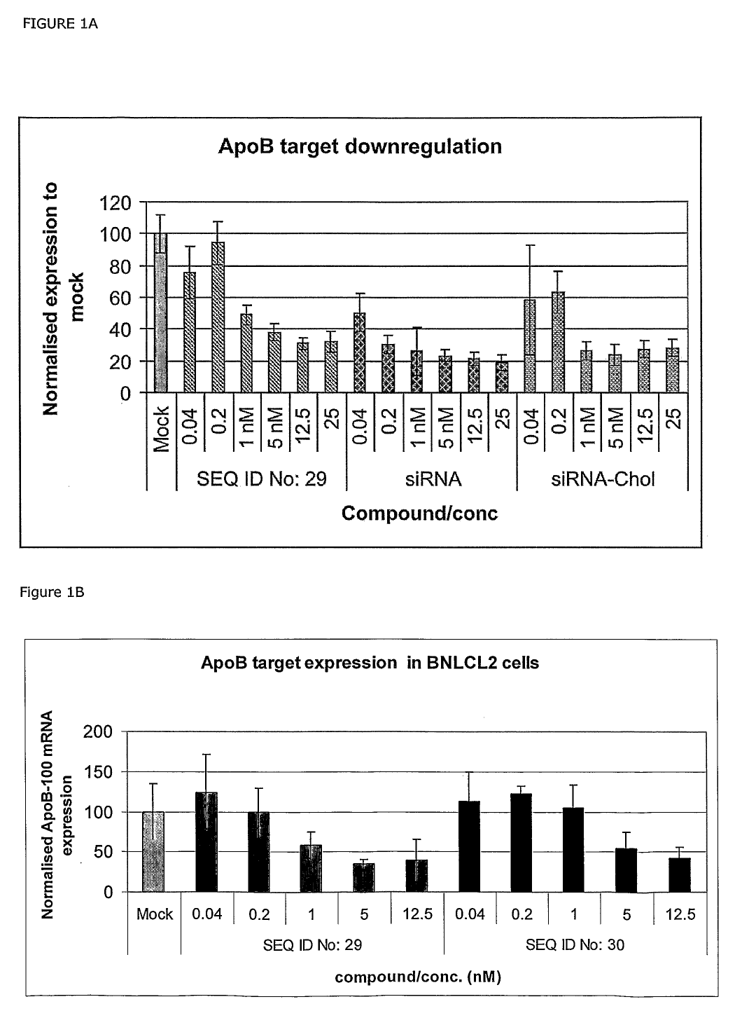

FIG. 1 demonstrates that siRNA and single stranded antisense oligonucleotides comprising LNA nucleotide analogues are potent in the same nanomolar range in vitro. However in vivo the 16-mer LNA antisense oligonucleotides of the invention are superior to both unmodified and cholesterol conjugated siRNA.

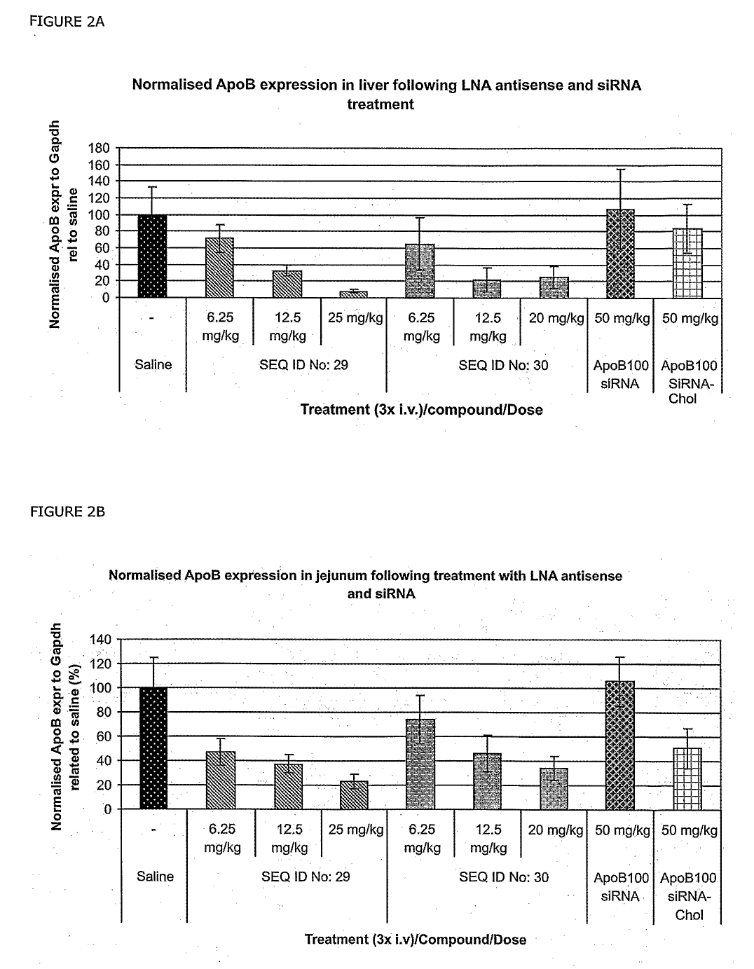

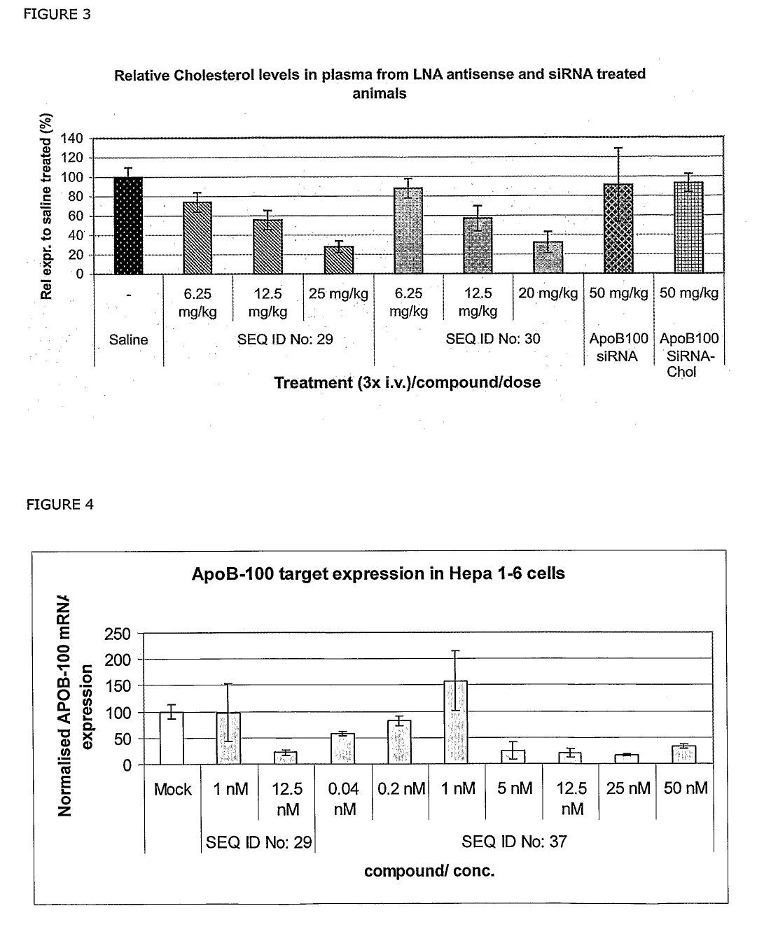

FIGS. 2A and 2B show LNA oligonucleotides of the invention which are up to 8-fold more potent than cholesteryl conjugated siRNA in vivo (cf.). LNA oligonucleotides lowered total cholesterol in mouse plasma while siRNA treatment did not (FIG. 3). Furthermore, LNA oligonucleotides are more biostable than siRNA.

Oligomeric compounds, which modulate expression of the target, are identified through experimentation or though rational design based on sequence information on the target and know-how on how best to design an oligonucleotide compound against a desired target. The sequences of these compounds are preferred embodiments of the invention. Likewise, the sequence motifs in the target to which these preferred oligomeric compounds are complementary (referred to as “hot spots”) are preferred sites for targeting.

Oligomeric Compounds and Oligonucleotide Compounds

The terms “Oligomeric compound”, which is interchangeable with the term “oligonucleotide”, “oligo”, and “oligonucleotide compound”, refer, in the context of the present invention, to an oligomer, i.e. a nucleic acid polymer (e.g. ribonucleic acid (RNA) or deoxyribonucleic acid (DNA)) or nucleic acid analogue of those known in the art, preferably Locked Nucleic Acid (LNA), or a mixture thereof). This term includes oligonucleotides composed of naturally occurring nucleobases, sugars and internucleoside (backbone) linkages as well as oligonucleotides having non-naturally-occurring portions which function similarly or with specific improved functions. Fully or partly modified or substituted oligonucleotides are often preferred over native forms because of several desirable properties of such oligonucleotides, such as for instance, the ability to penetrate a cell membrane, good resistance to extra- and intracellular nucleases, high affinity and specificity for the nucleic acid target. The LNA analogue is particularly preferred, for example, regarding the above-mentioned properties. Therefore, in a highly preferable embodiment, the terms “oligomeric compound”, “oligonucleotide”, “oligo” and “oligonucleotide compound” according to the invention, are compounds which are built up of both nucleotide and nucleotide analogue units, such as LNA units to form a polymeric compound of between 12-50 nucleotides/nucleotide analogues (oligomer).

By the term “unit” is understood a monomer.

The oligomeric compounds of the invention are capable of hybridizing to either the apolipoprotein B messenger RNA(s) and/or the sense or complementary mammalian apolipoprotein B (Apo-B) DNA strands. NCBI Accession No. NM_{—}000384 provides an mRNA sequence for human apolipoprotein B. It is highly preferably that the oligomeric compound of the invention is capable of hybridising to the human apolipoprotein encoded by the nucleic acid disclosed in NCBI Accession No. NM_{—}000384, or reverse complement thereof, including, in a preferred embodiment, mRNA nucleic acid targets derived from said human apolipoprotein.

In a preferred embodiment, the oligonucleotides are capable of hybridising against the target nucleic acid, such as an ApoB mRNA, to form a duplex with a Tm of at least 37° C., such as at least 40° C., at least 50° C., at least 55° C., or at least 60° C. In one aspect the Tm is between 37° C. and 80° C., such as between 50 and 70° C.

Measurement of Tₘ

A 3 μM solution of the compound in 10 mM sodium phosphate/100 mM NaCl/0.1 nM EDTA, pH 7.0 is mixed with its complement DNA or RNA oligonucleotide at 3 μM concentration in 10 mM sodium phosphate/100 mM NaCl/0.1 nM EDTA, pH 7.0 at 90° C. for a minute and allowed to cool down to room temperature. The melting curve of the duplex is then determined by measuring the absorbance at 260 nm with a heating rate of 1° C./min. in the range of 25 to 95° C. The Tₘ is measured as the maximum of the first derivative of the melting curve.

The oligomeric compounds are preferably antisense oligomeric compounds, also referred to as ‘antisense oligonucleotides’ and ‘antisense inhibitors’.

Such antisense inhibitors, are compounds which comprise complementary nucleotide/nucleotide analogue sequences to the target nucleic acid, and may take the form of “siRNA”, “miRNA”, “ribozymes”, oligozymes”. However, preferably, the antisense inhibitors are single stranded oligonucleotides. The single stranded oligonucleotides are preferably complementary to the corresponding region of the target nucleic acid.

Typically, single stranded ‘antisense’ oligonucleotides specifically interact with the mRNA of the target gene, causing either targeted degradation of the mRNA, for example via the RNaseH mechanism, or otherwise preventing translation.

In one embodiment the oligomeric compound according to the invention may target the DNA encoding mammalian ApoB, such as the sense or antisense DNA strand. siRNAs are known to be able to interact with target DNA.

The oligomeric compound according to the invention preferably comprises at least three nucleotide analogues. The at least three nucleotide analogues are preferably locked nucleic acid nucleotide analogues, and the oligomeric compound which comprises such nucleotide analogues are referred to herein as “LNA oligomeric compound”, “LNA oligonucleotide compound” and “LNA oligonucleotide”.

Suitably, the terms “oligonucleotide compound”, “oligomeric compound”, “LNA oligomeric compound”, according to the invention, are oligonucleotides, as defined herein, which can induce a desired therapeutic effect in humans through for example binding by hydrogen bonding to a target nucleic acid.

The invention is directed to an oligomeric compound, such as an oligonucleotide, consisting of 8-50, such as 10-50, in particular 12-50 or 12-25, nucleotides and/or nucleotide analogues, wherein said compound comprises a subsequence of at least 8, e.g. at least 10, such as at least 12, such as at least 14, such as at least 15, such as 14, 15, 16 or 17, nucleotides or nucleotide analogues, said subsequence being located within (i.e. corresponding to) a sequence of the Apo-B100 and/or Apo-B48, nucleic acid target sequence. The nucleotide analogues are analogues of their respective nucleotides of the sequence SEQ ID NOS: 2-26, in particular SEQ ID NOS: 2, 3, 10, 11 and 21. Thus, the subsequence of the compound of the invention is located within (i.e. corresponds to) a sequence selected from the group consisting of SEQ ID NOS: 2-26, in particular SEQ ID NOS: 2, 3, 10, 11 and 21, or comprise analogues of the nucleotides within the sequence of SEQ ID NOS: 2-26, in particular SEQ ID NO: 2, 3, 10, 11, and 21.

Preferred groups of sequences which the subsequence of the compound is located within (or the subsequence comprises analogues of the nucleotides within) include SEQ ID NO: 2 & 3; SEQ ID NO: 2 & 3 & 11; SEQ ID NO: 10 & 11; SEQ ID No 21.

In one embodiment, the group of sequences which the subsequence of the compound is located within (or the subsequence comprises analogues of the nucleotides within) SEQ ID No 3.

In one embodiment, the group of sequences which the subsequence of the compound is located within (or the subsequence comprises analogues of the nucleotides within) a sequence selected from the group consisting of: SEQ ID No 2, SEQ ID No 3, SEQ ID No 6, SEQ ID No 7, SEQ ID No 8, SEQ ID No 9, SEQ ID No 10, SEQ ID No 11, SEQ ID No 12, SEQ ID No 13, SEQ ID No 14, SEQ ID No 15, SEQ ID No 16, SEQ ID No 17, SEQ ID No 27, SEQ ID No 28, SEQ ID No 48 and SEQ ID No 50.

In an interesting embodiment, the compound of the invention comprises from 8-50 nucleotides, wherein said compound comprises a subsequence of at least 8 nucleotides, said subsequence being located within a sequence selected from the group consisting of SEQ ID NOs: 2 and 3, wherein at least one nucleotide is replaced by a corresponding nucleotide analogue and wherein the 3′ end comprises nucleotide, rather than a nucleotide analogue.

In embodiments of the compound of the invention comprising from 8-50 nucleotides, wherein said compound comprises a subsequence of at least 8 nucleotides, said subsequence being located within a sequence selected from the group consisting of SEQ ID NOS: 2 and 3 and said nucleotides comprising LNA nucleotide analogues, the subsequence typically may comprise a stretch of 2-6 LNAs, as defined herein, followed by a stretch of 4-12 nucleotides, which is followed by a stretch of 2-6 LNAs, as defined herein.

The terms “located within” and “corresponding to”/“corresponds to” refer to the comparison between the combined sequence of nucleotides and nucleotide analogues of the oligomeric compound of the invention, or subsequence thereof, and the equivalent nucleotide sequence of i) the reverse complement of a Apolipoprotein B nucleic acid sequence (i.e. the nucleic acid target), and/or ii) the sequence of nucleotides provided in the group consisting of SEQ ID NOS: 2-26, and 59-67 respectfully (i.e. a sequence motif), or in one embodiment the reverse compliments thereof. Nucleotide analogues are compared directly to their equivalent nucleotides.

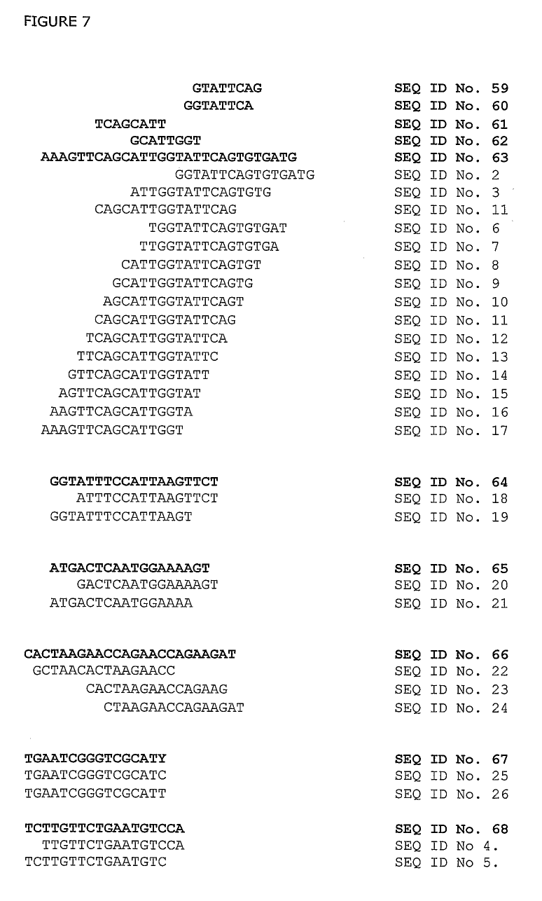

The subsequence may comprise at least 8, such as at least 9, such as at least 10, such as at least 11, such as at least 12, such as at least 13, such as at least 14, such as at least 15, such as at least 16, such as at least 17, such as at least 18, such as at least 19, or at least 20 nucleotides or nucleotide analogues which correspond to an equivalent number of consecutive nucleotides present in a nucleic acid selected from the group consisting of: SEQ ID No. 63, SEQ ID No. 64, SEQ ID No. 65, SEQ ID No. 66, SEQ ID No. 67 and SEQ ID No 68. (See FIG. 7).

Preferably, at least 3 nucleotide analogues are located within said subsequence, optionally as a consecutive sequence of at least 3 nucleotide analogues, such as a consecutive sequence of 3, 4, 5 or 6 nucleotide analogues.

In one preferred embodiment the oligomeric compound consists only of a subsequence, i.e. the entire sequence of the oligomeric compound is found in the corresponding sequence, such as a sequence selected from the group consisting of SEQ ID No 2-26 and SEQ ID No 59-62.

Preferably, there are no nucleotide or nucleotide analogues which form a mismatch when correlated to the corresponding region of the ApoB target sequence, i.e. all nucleotides and nucleotide analogues present in the oligomer of the invention are capable of forming consecutive base pairing with the ApoB nucleic acid target sequence.

However, in one embodiment there may be one mis-match or two mis-matches within a subsequence and the nucleic acid target sequence. When mismatches occur, it may be preferred that they are not between a nucleotide analogue and the target sequence.

However, in a ‘gap’ of a gapmer, which is capable of recruiting RNaseH, mismatches may lead to loss of the ability to recruit RNaseH. Typically 5 or 6 consecutive complementary nucleotides are required to ensure sufficient RNaseH activity.

In a preferable embodiment the oligonucleotide compound according to the invention comprises a sequence which corresponds to a SEQ ID NO 59. and/or SEQ ID NO. 60, wherein said subsequence may, optionally, comprise one or two mismatches.

In an embodiment, the oligonucleotide compound according to the invention comprises a sequence which corresponds to a SEQ ID NO 61. and/or SEQ ID NO. 62, wherein said subsequence may, optionally, comprise one or two mismatches.

In a preferable embodiment of the invention, the subsequence comprises of at least 8, such as at least 10, or at least 12, such as at least 14, such as 14, 15, 16, 17, 18, 19 or 20 nucleotides or nucleotide analogues which are located within (i.e. corresponding to) the equivalent number of consecutive nucleotides in SEQ ID No 63, wherein said subsequence may, optionally, comprise one or two mismatches.

In further embodiments of the invention, the subsequence comprises of at least 8, such as at least 10, or at least 12, such as at least 14, such as between 14 and 20, such as 14, 15, 16, 17, 18, 19 or 20 nucleotides or nucleotide analogues which are located within (i.e. corresponding to) the equivalent number of consecutive nucleotides in a nucleotide sequence selected from the group consisting of: SEQ ID No 64, SEQ ID No 65, SEQ ID No 66, SEQ ID No 67 and SEQ ID No 68, wherein said subsequence may, optionally, comprise one or two mismatches.

In one embodiment the oligomeric compound according to the invention is a double stranded oligonucleotide, wherein each strand comprises (or consists of) a total of 16-30 nucleotides and/or nucleotide analogues. It should be understood that the one strand of the double-stranded complex (oligonucleotide) corresponds to the oligonucleotide compound defined herein, and that the other strand is an oligonucleotide having a complementary sequence.

The total of, for example, 8-50 nucleotides and/or nucleotide analogues is intended to mean 8-50 nucleotides or 8-50 nucleotide analogues or a combination thereof not exceeding a combined total of 50 nucleoside units.

The compounds preferably consists of from 12-25 nucleotides or nucleotide analogues, such as 13, 14, 15, 16, 17, 18, 19, 20, 21, 22, 23, or 24 nucleotides or nucleotide analogues, such as between 15 and 22 nucleotides or nucleotide analogues, such as between 14 and 18 nucleotides or nucleotide analogues, more preferred 15 or 16 nucleotides or nucleotide analogues.

In the present context, the terms “nucleoside” and “nucleotide” are used in their normal meaning. For example, it contains a 2-deoxyribose unit which is bonded through its number one carbon atom to one of the nitrogenous bases adenine (A), cytosine (C), thymine (T) or guanine (G).

In a similar way, the term “nucleotide” means, for example in a preferred embodiment when relating to the compound of the invention the term “nucleotide” refers to a 2-deoxyribose unit which is bonded through its number one carbon atom to one of the nitrogenous bases adenine (A), cytosine (C), thymine (T) or guanine (G), and which is bonded through its number five carbon atom to an internucleoside phosphate (or in one embodiment an equivalent, such as a phosphorothioate group), or to a terminal group. A nucleotide may also, for example in one embodiment comprise of a ribose unit, such as a RNA nucleotide.

When used herein, the term “nucleotide analogue” refers to a non-natural occurring nucleotide wherein, for example in one preferred embodiment, either the ribose unit is different from 2-deoxyribose and/or the nitrogenous base is different from A, C, T and G and/or the internucleoside phosphate linkage group is different. Specific examples of nucleoside analogues are described by e.g. Freier & Altmann; Nucl. Acid Res., 1997, 25, 4429-4443 and Uhlmann; Curr. Opinion in Drug Development, 2000, 3(2), 293-213, and in Schemes 1

The terms “corresponding nucleoside/nucleotide analogue” and “corresponding nucleoside/nucleotide” are intended to indicate that the nitrogenous base in the nucleoside/nucleotide analogue and the nucleoside/nucleotide is identical. For example, when the 2-deoxyribose unit of the nucleotide is linked to an adenine, the “corresponding nucleoside analogue” contains a pentose unit (different from 2-deoxyribose) linked to an adenine.

The term “nucleic acid” is defined as a molecule formed by covalent linkage of two or more nucleotides. The terms “nucleic acid” and “polynucleotide” are used interchangeable herein. For example, DNA and RNA are nucleic acids.

The term “nucleic acid analogue” refers to a non-naturally occurring nucleic acid binding compound, i.e. in a preferred embodiment a compound, such as a sequence of at least one nucleotide and at least one nucleotide analogue, such as a LNA unit. Such compounds are not found naturally within the mammalian organism (or, in one embodiment were not publicly known to be found within the mammalian organism at the time of the invention).

A preferred nucleotide analogue is LNA, such as beta-D-oxy-LNA, alpha-L-oxy-LNA, beta-D-amino-LNA and beta-D-thio-LNA, most preferred beta-D-oxy-LNA. The compounds of the invention are typically those wherein said nucleotides comprise a linkage group selected from the group consisting of a phosphate group, a phosphorothioate group and a boranophosphate group, the internucleoside linkage may be —O—P(O)₂—O—, —O—P(O,S)—O—, in particular a phosphate group and/or a phosphorothioate group. In a particular embodiment, all nucleotides comprise a phosphorothioate group. In one embodiment, some or all of the nucleotides are linked to each other by means of a phosphorothioate group. Suitably, all nucleotides are linked to each other by means of a phosphorothioate group.

The nucleotides are typically linked to each other by means of the linkage group.

Nucleotide analogues and nucleic acid analogues are described in e.g. Freier & Altmann (Nucl. Acid Res., 1997, 25, 4429-4443) and Uhlmann (Curr. Opinion in Drug & Development (2000, 3(2): 293-213). Schemes 1 and 2 illustrate selected examples of nucleotide analogues suitable for making nucleic acids:

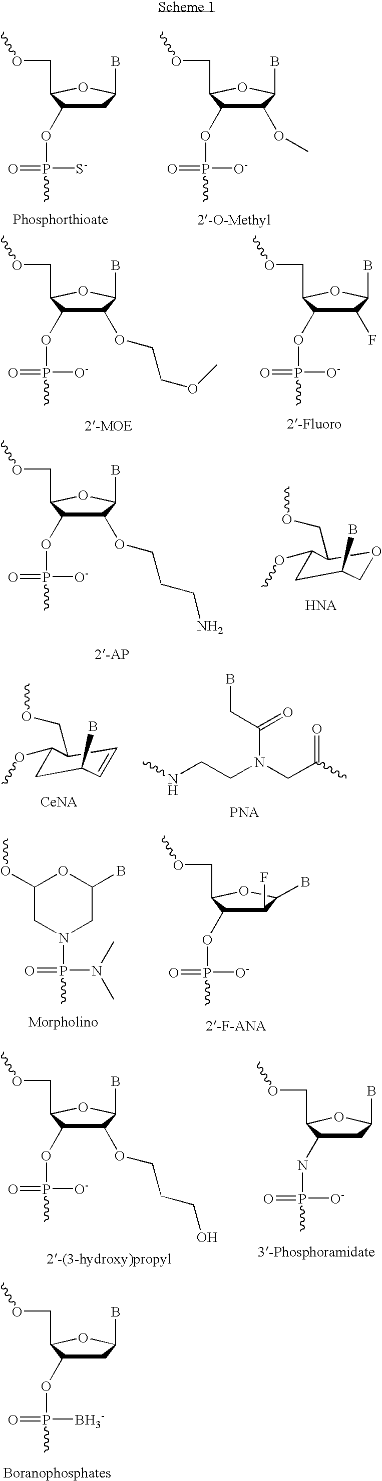

In an interesting embodiment, the compounds comprise of from 3-12 nucleotide analogues, e.g. 6 or 7 nucleotide analogues. In the by far most preferred embodiments, at least one of said nucleotide analogues is a locked nucleic acid (LNA), such as at least two, or at least 3 or at least 4, or at least 5, or at least 6, or at least 7, or at least 8, or at least 9, or at least 10, or at least 11, of the nucleotide analogues may be LNA, in one embodiment all the nucleotides analogues may be LNA.

The term “LNA” refers to a nucleotide analogue containing one bicyclic nucleotide analogue, also referred to as a LNA monomer.

The term “LNA” when used in the context of a “LNA oligonucleotides” refers to an oligonucleotide containing one or more bicyclic nucleoside analogues. The Locked Nucleic Acid (LNA) used in the oligonucleotide compounds of the invention has the structure of the general formula

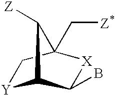

X and Y are independently selected among the groups —O—, —S—, —N(H)—, N(R)—, —CH₂— or —CH— (if part of a double bond), —CH₂—O—, —CH₂—S—, —CH₂—N(H)—, —CH₂—N(R)—, —CH₂—CH₂— or —CH₂—CH— (if part of a double bond), —CH═CH—, where R is selected form hydrogen and C₁₋₄-alkyl; Z and Z* are independently selected among an internucleoside linkage, a terminal group or a protecting group; B constitutes a natural or non-natural nucleobase; and the asymmetric groups may be found in either orientation.

Preferably, the Locked Nucleic Acid (LNA) used in the oligonucleotide compound of the invention comprises at least one Locked Nucleic Acid (LNA) unit according any of the formulas

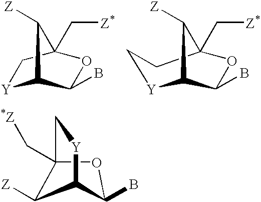

wherein Y is —O—, —S—, —NH—, or N(R^{H}); Z and Z* are independently selected among an internucleoside linkage, a terminal group or a protecting group; B constitutes a natural or non-natural nucleobase, and R^{H} is selected form hydrogen and C₁₋₄-alkyl.

Preferably, the Locked Nucleic Acid (LNA) used in the oligonucleotide compound of the invention comprises at internucleoside linkages selected from the group consisting of —O—P(O)₂—O—, —O—P(O,S)—O—, —O—P(S)₂—O—, —S—P(O)₂—O—, —S—P(O,S)—O—, —S—P(S)₂—O—, —O—P(O)₂—S—, —O—P(O,S)—S—, —S—P(O)₂—S—, —O—PO(R^{H})—O—, O—PO(OCH₃)—O—, —O—PO(NRH)—O—, —O—PO(OCH₂CH₂S—R)—O—, —O—PO(BH₃)—O—, —O—PO(NHR^{H})—O—, —O—P(O)₂—NR^{H}—, —NR^{H}—P(O)₂—O—, —NR^{H}—CO—O—, where R^{H} is selected form hydrogen and C₁₋₄-alkyl.

As stated, in an interesting embodiment of the invention, the oligonucleotide compounds contain at least one unit of chemistry termed LNA (Locked Nucleic Acid).

Specifically preferred LNA units are shown in scheme 2.

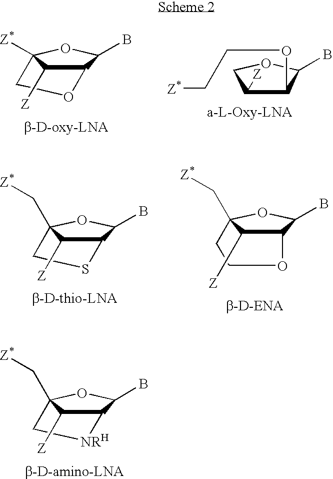

The term “thio-LNA” comprises a locked nucleotide in which at least one of X or Y in the general formula above is selected from S or —CH₂—S—. Thio-LNA can be in both beta-D and alpha-L-configuration.

The term “amino-LNA” comprises a locked nucleotide in which at least one of X or Y in the general formula above —N(H)—, N(R)—, CH₂—N(H)—, —CH₂—N(R)— where R is selected form hydrogen and C₁₋₄-alkyl. Amino-LNA can be in both beta-D and alpha-L-configuration.

The term “oxy-LNA” comprises a locked nucleotide in which at least one of X or Y in the general formula above represents —O— or —CH₂—O—. Oxy-LNA can be in both beta-D and alpha-L-configuration.

The term “ena-LNA” comprises a locked nucleotide in which Y in the general formula above is —CH₂—O— (where the oxygen atom of —CH₂—O— is attached to the 2′-position relative to the nucleobase B).

In a preferred embodiment LNA is selected from beta-D-oxy-LNA, alpha-L-oxy-LNA, beta-D-amino-LNA and beta-D-thio-LNA, in particular beta-D-oxy-LNA. The nucleosides and/or LNAs are typically linked together by means of phosphate groups and/or by means of phosphorothioate groups

The term “at least one” comprises the integers larger than or equal to 1, such as 1, 2, 3, 4, 5, 6, 7, 8, 9, 10, 11, 12, 13, 14, 15, 16, 17, 18, 19, 20, 21 and so forth.

As used herein, the term “target nucleic acid” encompasses DNA encoding the Apo-B100, RNA (including pre-mRNA and mRNA and mRNA edit) transcribed from such DNA, and also cDNA derived from such RNA.

The “target protein” is mammalian apolipoprotein B, preferably human apolipoprotein B. It will be recognised that as ApoB-100 and ApoB-48 both originate from the same genetic sequence, that the oligomeric compounds according to the invention may be used for down-regulation of either, or both forms of apolipoprotein B, and both ApoB-100 encoding mRNA, and the RNA edited form, which encodes Apo-B48.

As used herein, the term “gene” means the gene including exons, introns, non-coding 5′ and 3′ regions and regulatory elements and all currently known variants thereof and any further variants, which may be elucidated.

As used herein, the term “mRNA” means the presently known mRNA transcript(s) of a targeted gene, and any further transcripts, which may be identified.

As used herein, the term “modulation” means either an increase (stimulation) or a decrease (inhibition) in the expression of a gene. In the present invention, inhibition is the preferred form of modulation of gene expression and mRNA is a preferred target.

As used herein, the term “targeting” an antisense compound to a particular target nucleic acid means providing the antisense oligonucleotide to the cell, animal or human in such a way that the antisense compound are able to bind to and modulate the function of its intended target.

A preferred nucleotide analogue is LNA.

A further preferred nucleotide analogue is wherein the internucleoside phosphate linkage is a phosphorothioate.

A still further preferred nucleotide analogue is wherein the nucleotide is LNA with an internucleoside phosphorothioate linkage.

In an interesting embodiment, the 3′ end of the compound of the invention comprises a nucleotide, rather than a nucleotide analogue.

Preferably, the oligomeric compound, such as an antisense oligonucleotide, according to the invention comprises at least one Locked Nucleic Acid (LNA) unit, such as 3, 4, 5, 6, 7, 8, 9, or 10 Locked Nucleic Acid (LNA) units, preferably between 4 to 9 LNA units, such as 6-9 LNA units, most preferably 6, 7 or 8 LNA units. Preferably the LNA units comprise at least one beta-D-oxy-LNA unit(s) such as 4, 5, 6, 7, 8, 9, or 10 beta-D-oxy-LNA units. All the LNA units may be beta-D-oxy-LNA units, although it is considered that the oligomeric compounds, such as the antisense oligonucleotide, may comprise more than one type of LNA unit. Suitably, the oligomeric compound may comprise both beta-D-oxy-LNA, and one or more of the following LNA units: thio-LNA, amino-LNA, oxy-LNA, ena-LNA and/or alpha-LNA in either the D-beta or L-alpha configurations or combinations thereof.

In an embodiment of the compound of the invention which comprise nucleotide analogues, such as LNA nucleotide analogues, the subsequence typically may comprise a stretch of 2-6 nucleotide analogues, such as LNA nucleotide analogues, as defined herein, followed by a stretch of 4-12 nucleotides, which is followed by a stretch of 2-6 nucleotide analogues, such as LNA nucleotide analogues, as defined herein.

Subsequences comprising a stretch of nucleotide analogues, such as LNA nucleotide analogues, followed by a stretch of nucleotides, followed by a stretch of nucleotide analogues LNAs are known as gapmers.

Suitably, in one such “gapmer” embodiment, said subsequence comprises a stretch of 4 nucleotide analogues, such as LNA nucleotide analogues, as defined herein, followed by a stretch of 8 nucleotides, which is followed by a stretch of 4 nucleotide analogues, such as LNA nucleotide analogues as defined herein, optionally with a single nucleotide at the 3′ end.

In one further “gapmer” embodiment, said subsequence comprises a stretch of 3 nucleotide analogues, such as LNA nucleotide analogues, as defined herein, followed by a stretch of 9 nucleotides, which is followed by a stretch of 3 nucleotide analogues, such as LNA nucleotide analogues as defined herein, optionally with a single nucleotide at the 3′ end. Such a design has surprisingly been found to be very effective.

In one further “gapmer” embodiment, said subsequence comprises a stretch of 4 nucleotide analogues, such as LNA nucleotide analogues, as defined herein, followed by a stretch of 8 nucleotides, which is followed by a stretch of 3 nucleotide analogues, such as LNA nucleotide analogues as defined herein, optionally with a single nucleotide at the 3′ end.

Preferably, the oligomeric compound, such as an antisense oligonucleotide, may comprise both LNA and DNA units. Preferably the combined total of LNA and DNA units is between 14-20, such as between 15-18, more preferably 16 or 17 LNA/DNA units. Preferably the ratio of LNA to DNA present in the oligomeric compound of the invention is between 0.3 and 1, more preferably between 0.4 and 0.9, such as between 0.6 and 0.8.

Preferably the oligomeric compound, such as an antisense oligonucleotide, according to the invention is a gapmer, comprising a polynucleotide sequence of formula (5′ to 3′), A-B-C (and optionally D), wherein; A (5′ region) consists or comprises of at least one LNA unit, such as between 1-6 LNA units, preferably between 2-5 LNA units, most preferably 4 LNA units and; B (central domain), preferably immediately 3′ to A, consists or comprises at least one DNA sugar unit, such as 1-12 DNA units, preferably between 4-12 DNA units, more preferably between 6-10 DNA units, such as between 7-9DNA units, most preferably 8 DNA units, and; C(3′ region) preferably immediately 3′ to B, consists or comprises at of at least one LNA unit, such as between 1-6 LNA units, preferably between 2-5 LNA units, most preferably 4 LNA units. Preferred gapmer designs are disclosed in WO2004/046160.

In a gapmer oligonucleotide, it is preferable that any mismatches are not within the central domain (C) above, which preferably comprises or consists of DNA units. For RNAse H digestion it is typically found at least 5 consecutive nucleotides (or analogues which are capable of recruiting RNaseH to the oligo/target hybrid) are required in the central domain. Therefore, for gapmers, where the central domain exceeds 5 consecutive nucleotides, it is envisaged that one, or possibly two mismatches may be acceptable, although not preferable.

In one embodiment of gapmer oligonucleotides, it may be preferred that any mismatches are located towards the 5′ or 3′ termini of the gapmer. In such an embodiment, it is preferred that in a gapmer oligonucleotide which comprises mismatches with the target mRNA, that such mismatches are located either in 5′ and/or 3′ regions, and/or said mismatches are between the 5′ or 3′ terminal nucleotide unit of said gapmer oligonucleotide and target molecule.

In one embodiment, the gapmer, of formula A-B-C, further comprises a further region, D, which consists or comprises, preferably consists, of one or more DNA sugar residue terminal of the 3′ region (C) of the oligomeric compound, such as between one and three DNA sugar residues, including between 1 and 2 DNA sugar residues, most preferably 1 DNA sugar residue.

In one embodiment, within the oligomeric compound according to the invention, such as an antisense oligonucleotide, which comprises LNA, all LNA C residues are 5′methyl-Cytosine.

In one particularly interesting embodiment, the compound has the formula

5′-[(LNA)₃₋₄-(DNA/RNA)₈₋₉-(LNA)₃-(DNA/RNA)₁]-3′

wherein “LNA” designates an LNA nucleotide and “DNA” and “RNA” designate a deoxyribonucleotide and a ribonucleotide, respectively.

More particular, the compound may be selected from the group consisting of SEQ ID NOS: 29, 30, 31, 32, 33, 34, 35, 36, 37, 38, 39, 40, 41, 42, 43, 44, 45, 46 and 47. Preferred compounds may be selected from the group consisting of SEQ ID No 29, 30, 31, 32, 36, 37, 38, 40, 41 and 42, or from the group consisting of SEQ ID No 30 and 31, and/or from the group consisting of SEQ ID No 36, 37 and 38, and/or from the group consisting of SEQ ID No 41 and 42. Currently most preferred compounds are those of selected from the group consisting of SEQ ID NO: 29, SEQ ID NO: 30 and SEQ ID NO: 37.

Suitably, said nucleotides and/or said LNAs may be linked together by means of phosphate groups and/or Phosphorothioate groups of combinations thereof.

In one embodiment, said nucleotides and/or said LNAs are preferably linked together by means of phosphorothioate groups.

In one embodiment, the invention provides for a oligonucleotide compound comprising or consisting of SEQ ID NO: 29

In one embodiment, the invention provides for a oligonucleotide compound comprising or consisting of SEQ ID NO: 30

In one embodiment, the invention provides for a oligonucleotide compound comprising or consisting of SEQ ID NO: 31

In one embodiment, the invention provides for a oligonucleotide compound comprising or consisting of SEQ ID NO: 32

In one embodiment, the invention provides for a oligonucleotide compound comprising or consisting of SEQ ID NO: 33

In one embodiment, the invention provides for a oligonucleotide compound comprising or consisting of SEQ ID NO: 34

In one embodiment, the invention provides for a oligonucleotide compound comprising or consisting of SEQ ID NO: 35

In one embodiment, the invention provides for a oligonucleotide compound comprising or consisting of SEQ ID NO: 36

In one embodiment, the invention provides for the oligonucleotide compound comprising or consisting of SEQ ID NO: 37

In one embodiment, the invention provides for the oligonucleotide compound comprising or consisting of SEQ ID NO: 38

In one embodiment, the invention provides for the oligonucleotide compound comprising or consisting of SEQ ID NO: 39

In one embodiment, the invention provides for the oligonucleotide compound comprising or consisting of SEQ ID NO: 40

In one embodiment, the invention provides for the oligonucleotide compound comprising or consisting of SEQ ID NO: 41

In one embodiment, the invention provides for the oligonucleotide compound comprising or consisting of SEQ ID NO: 42

In one embodiment, the invention provides for the oligonucleotide compound comprising or consisting of SEQ ID NO: 43

In one embodiment, the invention provides for the oligonucleotide compound comprising or consisting of SEQ ID NO: 44

In one embodiment, the invention provides for the oligonucleotide compound comprising or consisting of SEQ ID NO: 45

In one embodiment, the invention provides for the oligonucleotide compound comprising or consisting of SEQ ID NO: 46

In one embodiment, the invention provides for the oligonucleotide compound comprising or consisting of SEQ ID NO: 47

In one embodiment, when the oligonucleotide according to the invention is an RNA oligonucleotide, such as SEQ IDs No 48, 49, 50 or 51, the 3′ terminal contains two co-joined 2′-O-methyl-modified ribonucleotide units, immediately adjacent to the terminal ribonucleotide.

Preparation of Oligonucleotide Compounds

The LNA nucleotide analogue building blocks (β-D-oxy-LNA, β-D-thio-LNA, β-D-amino-LNA and α-L-oxy-LNA) can be prepared following published procedures and references cited therein, see, e.g., WO 03/095467 A1; D. S. Pedersen, C. Rosenbohm, T. Koch (2002) Preparation of LNA Phosphoramidites, Synthesis 6, 802-808; M. D. Sørensen, L. Kvaernø, T. Bryld, A. E. H\dot{a}kansson, B. Verbeure, G. Gaubert, P. Herdewijn, J. Wengel (2002) α-L-ribo-configured Locked Nucleic Acid (α-l-LNA): Synthesis and Properties, J. Am. Chem. Soc., 124, 2164-2176; S. K. Singh, R. Kumar, J. Wengel (1998) Synthesis of Novel Bicyclo[2.2.1] Ribonucleosides: 2′-Amino- and 2′-Thio-LNA Monomeric Nucleosides, J. Org. Chem. 1998, 63, 6078-6079; C. Rosenbohm, S. M. Christensen, M. D. Sørensen, D. S. Pedersen, L. E. Larsen, J. Wengel, T. Koch (2003) Synthesis of 2′-amino-LNA: a new strategy, Org. Biomol. Chem. 1, 655-663; and WO 2004/069991 A2.

One particular example of a thymidine LNA monomer is the (1S,3R,4R,7S)-7-hydroxy-1-hydroxymethyl-3-(thymin-1yl)-2,5-dioxa-bicyclo[2:2:1]heptane.

The LNA oligonucleotides can be prepared as described in the Examples and in WO 99/14226, WO 00/56746, WO 00/56748, WO 00/66604, WO 00/125248, WO 02/28875, WO 2002/094250 and WO 03/006475. Thus, the LNA oligonucleotides may be produced using the oligomerisation techniques of nucleic acid chemistry well-known to a person of ordinary skill in the art of organic chemistry. Generally, standard oligomerisation cycles of the phosphoramidite approach (S. L. Beaucage and R. P. Iyer, Tetrahedron, 1993, 49, 6123; S. L. Beaucage and R. P. Iyer, Tetrahedron, 1992, 48, 2223) are used, but e.g. H-phosphonate chemistry, phosphotriester chemistry can also be used.

For some monomers, longer coupling time, and/or repeated couplings and/or use of more concentrated coupling reagents may be necessary or beneficial.

The phosphoramidites employed couple typically with satisfactory >95% step-wise yields. Oxidation of the Phosphorous(III) to Phosphorous(V) is normally done with e.g. iodine/pyridine/H₂O. This yields after deprotection the native phosphorodiester internucleoside linkage. In the case that a phosphorothioate internucleoside linkage is prepared a thiolation step is performed by exchanging the normal, e.g. iodine/pyridine/H₂O, oxidation used for synthesis of phosphorodiester internucleoside linkages with an oxidation using the ADTT reagent (xanthane hydride (0.01 M in acetonitrile:pyridine 9:1; v/v)). Other thiolation reagents are also possible to use, such as Beaucage and PADS. The phosphorothioate LNA oligonucleotides were efficiently synthesized with stepwise coupling yields >=98%.

LNA oligonucleotides comprising β-D-amino-LNA, β-D-thio-LNA, and/or α-L-LNA can also efficiently be synthesized with step-wise coupling yields ≧98% using the phosphoramidite procedures.

Purification of LNA oligonucleotides was can be accomplished using disposable reversed phase purification cartridges and/or reversed phase HPLC and/or precipitation from ethanol or butanol. Capillary gel electrophoresis, reversed phase HPLC, MALDI-MS, and ESI-MS were used to verify the purity of the synthesized LNA oligonucleotides.

Salts

The LNA oligonucleotides can be employed in a variety of pharmaceutically acceptable salts. As used herein, the term refers to salts that retain the desired biological activity of the LNA oligonucleotide and exhibit minimal undesired toxicological effects. Non-limiting examples of such salts can be formed with organic amino acid and base addition salts formed with metal cations such as zinc, calcium, bismuth, barium, magnesium, aluminum, copper, cobalt, nickel, cadmium, sodium, potassium, and the like, or with a cation formed from ammonia, N,N-dibenzylethylene-diamine, D-glucosamine, tetraethylammonium, or ethylenediamine; or combinations, e.g., a zinc tannate salt or the like.

Such salts are formed, from the LNA oligonucleotides which possess phosphorodiester group and/or phosphorothioate groups, and are, for example, salts with suitable bases. These salts include, for example, nontoxic metal salts which are derived from metals of groups Ia, Ib, IIa and IIb of the Periodic System of the elements, in particular suitable alkali metal salts, for example lithium, sodium or potassium salts, or alkaline earth metal salts, for example magnesium or calcium salts. They furthermore include zinc and ammonium salts and also salts which are formed with suitable organic amines, such as unsubstituted or hydroxyl-substituted mono-, di- or tri-alkylamines, in particular mono-, di- or tri-alkylamines, or with quaternary ammonium compounds, for example with N-methyl-N-ethylamine, diethylamine, triethylamine, mono-, bis- or tris-(2-hydroxy-lower alkyl)amines, such as mono-, bis- or tris-(2-hydroxyethyl)amine, 2-hydroxy-tert-butylamine or tris(hydroxymethyl)methylamine, N,N-di-lower alkyl-N-(hydroxy-lower alkyl)amines, such as N,N-dimethyl-N-(2-hydroxyethyl)-amine or tri-(2-hydroxyethyl)amine, or N-methyl-D-glucamine, or quaternary ammonium compounds such as tetrabutylammonium salts. Lithium salts, sodium salts, magnesium salts, zinc salts or potassium salts are preferred, with sodium salts being particularly preferred.

Prodrugs

In one embodiment, the LNA oligonucleotide may be in the form of a prodrug. Oligonucleotides are by virtue negatively charged ions. Due to the lipophilic nature of cell membranes, the cellular uptake of oligonucleotides is reduced compared to neutral or lipophilic equivalents. This polarity “hindrance” can be avoided by using the prodrug approach (see e.g. Crooke, R. M. (1998) in Crooke, S. T. Antisense research and Application. Springer-Verlag, Berlin, Germany, vol. 131, pp. 103-140). In this approach, the LNA oligonucleotides are prepared in a protected manner so that the LNA oligonucleotides are neutral when it is administered. These protection groups are designed in such a way that they can be removed when the LNA oligonucleotide is taken up by the cells. Examples of such protection groups are S-acetylthioethyl (SATE) or S-pivaloylthioethyl (t-butyl-SATE). These protection groups are nuclease resistant and are selectively removed intracellulary.

Conjugates

A further aspect of the invention relates to a conjugate comprising the compound as defined herein at least one non-nucleotide or non-polynucleotide moiety covalently attached to said compound.

In a related aspect of the invention, the compound of the invention is linked to ligands so as to form a conjugates said ligands intended to increase the cellular uptake of the conjugate relative to the antisense oligonucleotides.

The compounds or conjugates of the invention may also be conjugated or further conjugated to active drug substances, for example, aspirin, ibuprofen, a sulfa drug, a cholesterol lowering agent, an antidiabetic, an antibacterial agent, a chemotherapeutic agent or an antibiotic.

In the present context, the term “conjugate” is intended to indicate a heterogenous molecule formed by the covalent attachment of an LNA oligonucleotide as described herein (i.e. a compound comprising a sequence of nucleosides and LNA nucleoside analogues) to one or more non-nucleotide or non-polynucleotide moieties.

Thus, the LNA oligonucleotides may, e.g., be conjugated or form chimera with non-nucleotide or non-polynucleotide moieties including Peptide Nucleic Acids (PNA), proteins (e.g. antibodies for a target protein), macromolecules, low molecular weight drug substances, fatty acid chains, sugar residues, glycoproteins, polymers (e.g. polyethylene glycol), micelle-forming groups, antibodies, carbohydrates, receptor-binding groups, steroids such as cholesterol, polypeptides, intercalating agents such as an acridine derivative, a long-chain alcohol, a dendrimer, a phospholipid and other lipophilic groups or combinations thereof, etc., just as the LNA oligonucleotides may be arranged in dimeric or dendritic structures. The LNA oligonucleotides or conjugates may also be conjugated or further conjugated to active drug substances, for example, aspirin, ibuprofen, a sulfa drug, an antidiabetic, an antibacterial agent, a chemotherapeutic compound or an antibiotic.

Conjugating in this way confers advantageous properties with regard to the pharmacokinetic characteristics of the LNA oligonucleotides. In particular, conjugating in this way achieves increased cellular uptake.

In one embodiment, an LNA oligonucleotide is linked to ligands so as to form a conjugate, said ligands intended to increase the cellular uptake of the conjugate relative to the antisense LNA oligonucleotides. This conjugation can take place at the terminal positions 5′/3′-OH but the ligands may also take place at the sugars and/or the bases. In particular, the growth factor to which the antisense LNA oligonucleotide may be conjugated, may comprise transferrin or folate. Transferrin-polylysine-oligonucleotide complexes or folate-polylysine-oligonucleotide complexes may be prepared for uptake by cells expressing high levels of transferrin or folate receptor. Other examples of conjugates/ligands are cholesterol moieties, duplex intercalators such as acridine, poly-L-lysine, “end-capping” with one or more nuclease-resistant linkage groups such as phosphoromonothioate, and the like.

The preparation of transferrin complexes as carriers of oligonucleotide uptake into cells is described by Wagner et al., Proc. Nat. Acad. Sci. USA 87, 3410-3414 (1990). Cellular delivery of folate-macromolecule conjugates via folate receptor endocytosis, including delivery of an antisense oligonucleotide, is described by Low et al., U.S. Pat. No. 5,108,921. Also see, Leamon et al., Proc. Natl. Acad. Sci. 88, 5572 (1991).

Pharmaceutical Composition

A particularly interesting aspect of the invention is directed to a pharmaceutical composition comprising a compound as defined herein or a conjugate as defined herein, and a pharmaceutically acceptable diluent, carrier or adjuvant. In a particularly interesting embodiment, the pharmaceutical composition is adapted for oral administration.

Directions for the preparation of pharmaceutical compositions can be found in “Remington: The Science and Practice of Pharmacy” by Alfonso R. Gennaro, and in the following.

It should be understood that the present invention also particularly relevant for a pharmaceutical composition, which comprises a least one antisense oligonucleotide construct of the invention as an active ingredient. It should be understood that the pharmaceutical composition according to the invention optionally comprises a pharmaceutical carrier, and that the pharmaceutical composition optionally comprises further antisense compounds, chemotherapeutic agents, cholesterol lowering agents, anti-inflammatory compounds, antiviral compounds and/or immuno-modulating compounds.

As stated, the pharmaceutical composition of the invention may further comprise at least one therapeutic/prophylactic compound. The compound is typically selected from the group consisting of bile salt sequestering resins (e.g., cholestyramine, colestipol, and colesevelam hydrochloride), HMGCoA-reductase inhibitors (e.g., lovastatin, cerivastatin, pravastatin, atorvastatin, simvastatin, and fluvastatin), nicotinic acid, fibric acid derivatives (e.g., clofibrate, gemfibrozil, fenofibrate, bezafibrate, and ciprofibrate), probucol, neomycin, dextrothyroxine, plant-stanol esters, cholesterol absorption inhibitors (e.g., ezetimibe), implitapide, inhibitors of bile acid transporters (apical sodium-dependent bile acid transporters), regulators of hepatic CYP7a, estrogen replacement therapeutics (e.g., tamoxifen), and anti-inflammatories (e.g., glucocorticoids).

The oligonucleotide compound or conjugate comprised in this invention can be employed in a variety of pharmaceutically acceptable salts. As used herein, the term refers to salts that retain the desired biological activity of the herein identified compounds and exhibit minimal undesired toxicological effects, cf. “Conjugates”

In one embodiment of the invention the oligonucleotide compound or conjugate may be in the form of a prodrug, cf. “Prodrugs”.

The invention also includes the formulation of one or more oligonucleotide compound or conjugate as disclosed herein. Pharmaceutically acceptable binding agents and adjuvants may comprise part of the formulated drug. Capsules, tablets and pills etc. may contain for example the following compounds: microcrystalline cellulose, gum or gelatin as binders; starch or lactose as excipients; stearates as lubricants; various sweetening or flavouring agents. For capsules the dosage unit may contain a liquid carrier like fatty oils. Likewise coatings of sugar or enteric agents may be part of the dosage unit. The oligonucleotide formulations may also be emulsions of the active pharmaceutical ingredients and a lipid forming a micellular emulsion. Such formulations are particularly useful for oral administration.

An oligonucleotide of the invention may be mixed with any material that do not impair the desired action, or with material that supplement the desired action. These could include other drugs including other nucleotide compounds.

For parenteral, subcutaneous, intradermal or topical administration the formulation may include a sterile diluent, buffers, regulators of tonicity and antibacterials. The active compound may be prepared with carriers that protect against degradation or immediate elimination from the body, including implants or microcapsules with controlled release properties. For intravenous administration the preferred carriers are physiological saline or phosphate buffered saline.

Preferably, an oligonucleotide compound is included in a unit formulation such as in a pharmaceutically acceptable carrier or diluent in an amount sufficient to deliver to a patient a therapeutically effective amount without causing serious side effects in the treated patient.

The pharmaceutical compositions of the present invention may be administered in a number of ways depending upon whether local or systemic treatment is desired and upon the area to be treated. Administration may be (a) oral (b) pulmonary, e.g., by inhalation or insufflation of powders or aerosols, including by nebulizer; intratracheal, intranasal, (c) topical including epidermal, transdermal, ophthalmic and to mucous membranes including vaginal and rectal delivery; or (d) parenteral including intravenous, intraarterial, subcutaneous, intraperitoneal or intramuscular injection or infusion; or intracranial, e.g., intrathecal or intraventricular, administration. In one embodiment the active LNA oligonucleotide is administered IV, IP, orally, topically or as a bolus injection or administered directly in to the target organ.

Pharmaceutical compositions and formulations for topical administration may include transdermal patches, ointments, lotions, creams, gels, drops, sprays, suppositories, liquids and powders. Conventional pharmaceutical carriers, aqueous, powder or oily bases, thickeners and the like may be necessary or desirable. Coated condoms, gloves and the like may also be useful. Preferred topical formulations include those in which the oligonucleotides of the invention are in admixture with a topical delivery agent such as lipids, liposomes, fatty acids, fatty acid esters, steroids, chelating agents and surfactants.

Compositions and formulations for oral administration include but are not restricted to powders or granules, microparticulates, nanoparticulates, suspensions or solutions in water or non-aqueous media, capsules, gel capsules, sachets, tablets or miniTablets. Typically,

Compositions and formulations for parenteral, intrathecal or intraventricular administration may include sterile aqueous solutions which may also contain buffers, diluents and other suitable additives such as, but not limited to, penetration enhancers, carrier compounds and other pharmaceutically acceptable carriers or excipients.

Pharmaceutical compositions of the present invention include, but are not limited to, solutions, emulsions, and liposome-containing formulations. These compositions may be generated from a variety of components that include, but are not limited to, preformed liquids, self-emulsifying solids and self-emulsifying semisolids. Delivery of drug to liver tissue may be enhanced by carrier-mediated delivery including, but not limited to, cationic liposomes, cyclodextrins, porphyrin derivatives, branched chain dendrimers, polyethylenimine polymers, nanoparticles and microspheres (Dass CR. J Pharm Pharmacol 2002; 54(1):3-27).

The pharmaceutical formulations of the present invention, which may conveniently be presented in unit dosage form, may be prepared according to conventional techniques well known in the pharmaceutical industry. Such techniques include the step of bringing into association the active ingredients with the pharmaceutical carrier(s) or excipient(s). In general the formulations are prepared by uniformly and intimately bringing into association the active ingredients with liquid carriers or finely divided solid carriers or both, and then, if necessary, shaping the product.

The compositions of the present invention may be formulated into any of many possible dosage forms such as, but not limited to, tablets, capsules, gel capsules, liquid syrups, soft gels and suppositories. The compositions of the present invention may also be formulated as suspensions in aqueous, non-aqueous or mixed media. Aqueous suspensions may further contain substances which increase the viscosity of the suspension including, for example, sodium carboxymethylcellulose, sorbitol and/or dextran. The suspension may also contain stabilizers.

LNA containing oligonucleotide compounds are useful for a number of therapeutic applications as indicated above. In general, therapeutic methods of the invention include administration of a therapeutically effective amount of an LNA-modified oligonucleotide to a mammal, particularly a human.

In a certain embodiment, the present invention provides pharmaceutical compositions containing (a) one or more antisense compounds and (b) one or more other cholesterol lowering agents which function by a non-antisense mechanism. When used with the compounds of the invention, such cholesterol lowering agents may be used individually (e.g. atorvastatin and oligonucleotide), sequentially (e.g. atorvastatin and oligonucleotide for a period of time followed by another agent and oligonucleotide), or in combination with one or more other such cholesterol lowering agents. All cholesterol lowering agents known to a person skilled in the art are here incorporated as combination treatments with compound according to the invention.

Anti-inflammatory drugs, including but not limited to nonsteroidal anti-inflammatory drugs and corticosteroids, antiviral drugs, and immuno-modulating drugs may also be combined in compositions of the invention. Two or more combined compounds may be used together or sequentially.

In another embodiment, compositions of the invention may contain one or more antisense compounds, particularly oligonucleotides, targeted to a first nucleic acid and one or more additional antisense compounds targeted to a second nucleic acid target. Two or more combined compounds may be used together or sequentially.

Dosing is dependent on severity and responsiveness of the disease state to be treated, and the course of treatment lasting from several days to several months, or until a cure is effected or a diminution of the disease state is achieved. Optimal dosing schedules can be calculated from measurements of drug accumulation in the body of the patient.

Optimum dosages may vary depending on the relative potency of individual oligonucleotides. Generally it can be estimated based on EC₅₀s found to be effective in in vitro and in vivo animal models. In general, dosage is from 0.01 μg to 1 g per kg of body weight, and may be given once or more daily, weekly, monthly or yearly, or even once every 2 to 10 years or by continuous infusion for hours up to several months. The repetition rates for dosing can be estimated based on measured residence times and concentrations of the drug in bodily fluids or tissues. Following successful treatment, it may be desirable to have the patient undergo maintenance therapy to prevent the recurrence of the disease state.

Method of Treatment

A person skilled in the art will appreciate that oligonucleotide compounds containing LNA can be used to combat apolipoprotein B (Apo-B100) linked diseases by many different principles, which thus falls within the spirit of the present invention.

The LNA oligonucleotide compounds may be designed as siRNA's which are small double stranded RNA molecules that are used by cells to silence specific endogenous or exogenous genes by an as yet poorly understood “antisense-like” mechanism.

It has been shown that β-D-oxy-LNA does not support RNaseH activity. However, this can be changed according to the invention by creating chimeric oligonucleotides composed of β-D-oxy-LNA and DNA, called gapmers. A gapmer is based on a central stretch of 4-12 nt DNA or modified monomers recognizable and cleavable by the RNaseH (the gap) typically flanked by 1 to 6 residues of β-D-oxy-LNA (the flanks). The flanks can also be constructed with LNA derivatives. There are other chimeric constructs according to the invention that are able to act via an RNaseH mediated mechanism. A headmer is defined by a contiguous stretch of β-D-oxy-LNA or LNA derivatives at the 5′-end followed by a contiguous stretch of DNA or modified monomers recognizable and cleavable by the RNaseH towards the 3′-end, and a tailmer is defined by a contiguous stretch of DNA or modified monomers recognizable and cleavable by the RNaseH at the 5′-end followed by a contiguous stretch of β-D-oxy-LNA or LNA derivatives towards the 3′-end. Other chimeras according to the invention, called mixmers consisting of an alternate composition of DNA or modified monomers recognizable and cleavable by RNaseH and β-D-oxy-LNA and/or LNA derivatives might also be able to mediate RNaseH binding and cleavage. Since α-L-LNA recruits RNaseH activity to a certain extent, smaller gaps of DNA or modified monomers recognizable and cleavable by the RNaseH for the gapmer construct might be required, and more flexibility in the mixmer construction might be introduced.

The clinical effectiveness of antisense oligonucleotides depends to a significant extent on their pharmacokinetics e.g. absorption, distribution, cellular uptake, metabolism and excretion. In turn these parameters are guided significantly by the underlying chemistry and the size and three-dimensional structure of the oligonucleotide.

Modulating the pharmacokinetic properties of an LNA oligonucleotide according to the invention may further be achieved through attachment of a variety of different moieties. For instance, the ability of oligonucleotides to pass the cell membrane may be enhanced by attaching for instance lipid moieties such as a cholesterol moiety, a thioether, an aliphatic chain, a phospholipid or a polyamine to the oligonucleotide. Likewise, uptake of LNA oligonucleotides into cells may be enhanced by conjugating moieties to the oligonucleotide that interacts with molecules in the membrane, which mediates transport into the cytoplasm.

The pharmacodynamic properties can according to the invention be enhanced with groups that improve oligomer uptake, enhance biostability such as enhance oligomer resistance to degradation, and/or increase the specificity and affinity of oligonucleotides hybridisation characteristics with target sequence e.g. a mRNA sequence.

The pharmaceutical composition according to the invention can be used for the treatment of conditions associated with abnormal levels of ApoB-100.

Examples of such conditions are hyperlipoproteinemia, familial type 3 hyperlipoproteinemia (familial dysbetalipoproteinemia), and familial hyperalphalipoprotienemia; hyperlipidemia, mixed hyperlipidemias, multiple lipoprotein-type hyperlipidemia, and familial combined hyperlipidemia; hypertriglyceridemia, familial hypertriglyceridemia, and familial lipoprotein lipase; hypercholesterolemia, statin-resistant hypercholesterolemia familial hypercholesterolemia, polygenic hypercholesterolemia, and familial defective apolipoprotein B; cardiovascular disorders including atherosclerosis and coronary artery disease; thrombosis; peripheral vascular disease; von Gierke's disease (glycogen storage disease, type I); lipodystrophies (congenital and acquired forms); Cushing's syndrome; sexual ateloitic dwarfism (isolated growth hormone deficiency); diabetes mellitus; hyperthyroidism; hypertension; anorexia nervosa; Werner's syndrome; acute intermittent porphyria; primary biliary cirrhosis; extrahepatic biliary 5 obstruction; acute hepatitis; hepatoma; systemic lupus erythematosis; monoclonal gammopathies (including myeloma, multiple myeloma, macroglobulinemia, and lymphoma); endocrinopathies; obesity; nephrotic syndrome; metabolic syndrome; inflammation; hypothyroidism; uremia (hyperurecemia); impotence; obstructive liver disease; idiopathic hypercalcemia; dysqlobulinemia; elevated insulin levels; Syndrome X; Dupuytren's contracture; AIDS; and Alzheimer's disease and dementia.

The invention also provides methods of reducing the risk of a condition comprising the step of administering to an individual an amount of compound of the invention sufficient to inhibit apolipoprotein B expression, said condition selected from pregnancy; intermittent claudication; gout; and mercury toxicity and amalgam illness. The invention further provides methods of inhibiting cholesterol particle binding to vascular endothelium comprising the step of administering to an individual an amount of a compound of the invention sufficient to inhibit apolipoprotein B expression, and as a result, the invention also provides methods of reducing the risk of: (i) cholesterol particle oxidization; (ii) monocyte binding to vascular endothelium; (iii) monocyte differentiation into macrophage; (iv) macrophage ingestion of oxidized lipid 30 particles and release of cytokines (including, but limited to IL-1, TNF-alpha, TGF-beta); (v) platelet formation of fibrous fibrofatty lesions and inflammation; (vi) endothelium lesions leading to clots; and (vii) clots leading to myocardial infarction or stroke, also comprising the step of administering to an individual an amount of a compound of the invention sufficient to inhibit apolipoprotein B expression.

The invention also provides methods of reducing hyperlipidemia associated with alcoholism, smoking, use of oral contraceptives, use of glucocorticoids, use of beta-adrenergic blocking agents, or use of isotretinoin (13-cis retinoic acid) comprising the step of administering to an individual an amount of a compound of the invention sufficient to inhibit apolipoprotein B expression.

The invention further provides use of a compound of the invention in the manufacture of a medicament for the treatment of any and all conditions disclosed herein.

Generally stated, one aspect of the invention is directed to a method of treating a mammal suffering from or susceptible to conditions associated with abnormal levels of ApoB-100, comprising administering to the mammal an therapeutically effective amount of an oligonucleotide targeted to Apo-B100 that comprises one or more LNA units.

An interesting aspect of the invention is directed to the use of a compound as defined herein or as conjugate as defined herein for the preparation of a medicament for the treatment of a condition according to above.

The methods of the invention are preferably employed for treatment or prophylaxis against diseases caused by abnormal levels of ApoB-100.

Furthermore, the invention described herein encompasses a method of preventing or treating a disease comprising a therapeutically effective amount of a Apo-B100 modulating oligonucleotide compound, including but not limited to high doses of the oligomer, to a human in need of such therapy. The invention further encompasses the use of a short period of administration of an Apo-B100 modulating oligonucleotide compound.

In one embodiment of the invention the oligonucleotide compound is linked to ligands/conjugates. It is way to increase the cellular uptake of antisense oligonucleotides.

Oligonucleotide compounds of the invention may also be conjugated to active drug substances, for example, aspirin, ibuprofen, a sulfa drug, an antidiabetic, an antibacterial or an antibiotic.

Alternatively stated, the invention is furthermore directed to a method for treating abnormal levels of ApoB-100, said method comprising administering a compound as defined herein, or a conjugate as defined herein or a pharmaceutical composition as defined herein to a patient in need thereof and further comprising the administration of a a further chemotherapeutic agent. Said further administration may be such that the further chemotherapeutic agent is conjugated to the compound of the invention, is present in the pharmaceutical composition, or is administered in a separate formulation.

The LNA containing oligonucleotide compounds of the present invention can also be utilized for as research reagents for diagnostics, therapeutics and prophylaxis. In research, the antisense oligonucleotides may be used to specifically inhibit the synthesis of Apo-B100 genes in cells and experimental animals thereby facilitating functional analysis of the target or an appraisal of its usefulness as a target for therapeutic intervention. In diagnostics the antisense oligonucleotides may be used to detect and quantitate Apo-B100 expression in cell and tissues by Northern blotting, in-situ hybridisation or similar techniques. For therapeutics, an animal or a human, suspected of having a disease or disorder, which can be treated by modulating the expression of Apo-B100 is treated by administering antisense compounds in accordance with this invention. Further provided are methods of treating an animal particular mouse and rat and treating a human, suspected of having or being prone to a disease or condition, associated with expression of Apo-B100 by administering a therapeutically or prophylactically effective amount of one or more of the antisense compounds or compositions of the invention.

The invention also relates to a compound or a conjugate as defined herein for use as a medicament.

The invention further relates to use of a compound or a conjugate as defined herein for the manufacture of a medicament for the treatment of abnormal levels of Apo-B100. Typically, said abnormal levels of Apo-B100 is in the form of atherosclerosis, hypercholesterolemia or hyperlipidemia.

Moreover, the invention relates to a method of treating a subject suffering from a disease or condition selected from atherosclerosis, hypercholesterolemia and hyperlipidemia, the method comprising the step of administering a pharmaceutical composition as defined herein to the subject in need thereof. Preferably, the pharmaceutical composition is administered orally.

### SOME EMBODIMENTS OF THE INVENTION

1. A compound consisting of a total of 12-50 nucleotides and/or nucleotide analogues, wherein said compound comprises a subsequence of at least 10 nucleotides or nucleotide analogues, said subsequence being located within a sequence selected from the group consisting of SEQ ID NOS: SEQ ID No 2, SEQ ID No 3, SEQ ID No 6, SEQ ID No 7, SEQ ID No 8, SEQ ID No 9, SEQ ID No 10, SEQ ID No 11, SEQ ID No 12, SEQ ID No 13, SEQ ID No 14, SEQ ID No 15, SEQ ID No 16, SEQ ID No 17, SEQ ID No 27, SEQ ID No 28, SEQ ID No 48 and SEQ ID No 50. wherein said compound comprises at least 3 nucleotide analogs.

2. A compound according to claim 1, consisting a double stranded oligonucleotide, wherein each strand comprises a total of 16-30 nucleotides and/or nucleotide analogues, wherein said compound comprises a subsequence of at least 10 nucleotides or nucleotide analogues, said subsequence being located within a sequence selected from SEQ ID NOS: 27, 28, (and/or 48 or 50) and, wherein said compound comprises at least 3 nucleotide analogs.

3. The compound according to embodiment 1 consisting of from 12-25 nucleotides or nucleotide analogs.

4. The compound according to embodiment 3 consisting of 15, 16, 17, 18, 19, 20, 21, or 22 nucleotides or nucleotide analogs.

5. The compound according to embodiment 4 consisting of 16 nucleotides or nucleotide analogs.

6. The compound according to any of embodiments 1-5, wherein said nucleotides comprise a linkage group selected from the group consisting of a phosphate group, a phosphorothioate group and a boranophosphate group, the internucleoside linkage may be —O—P(O)₂—O—, —O—P(O,S)—O—.

7. The compound according to embodiment 6, wherein said linkage is a phosphate group.

8. The compound according to embodiment 6, wherein said linkage is phosphorothioate group.

9. The compound according to embodiment 6, wherein all nucleotides comprise a phosphorothioate group.

10. The compound according to embodiment 9 comprising of from 3-12 nucleotide analogues.

11. The compound according to embodiment 10 comprising 6 nucleotide analogues.

12. The compound according to any of embodiments 10-11, wherein at least one of said nucleotide analogues is a locked nucleic acid (LNA).

13. The compound according to any of embodiment 12, wherein LNA is selected from beta-D-oxy-LNA, alpha-L-oxy-LNA, beta-D-amino-LNA or beta-D-thio-LNA.

14. The compound according to embodiment 13, wherein said nucleosides and/or LNAs are linked together by means of phosphate groups.

15. The compound according to embodiment 14, wherein said nucleosides and/or said LNAs are linked together by means of phosphorothioate groups.

16. The compound according to embodiment 12, wherein the subsequence is SEQ ID NO: 2.

17. The compound according to embodiment 12, wherein the subsequence is SEQ ID NO: 3.

18. The compound according to any of embodiments 16-17, wherein the 3′ end LNA is replaced by the corresponding natural nucleoside.

19. A compound consisting of SEQ ID No 29

20. A compound consisting of SEQ ID No 30

21. A conjugate comprising the compound according to any of embodiments 1-20 and at least one non-nucleotide or non-polynucleotide moiety covalently attached to said compound.

22. A pharmaceutical composition comprising a compound as defined in any of embodiments 1-20 or a conjugate as defined in embodiment 21, and a pharmaceutically acceptable diluent, carrier or adjuvant.

23. The pharmaceutical composition according to embodiment 22 further comprising at least one cholesterol-lowering compound.

24. The pharmaceutical composition according to embodiment 23, wherein said compound is selected from the group consisting of bile salt sequestering resins (e.g., cholestyramine, colestipol, and colesevelam hydrochloride), HMGCoA-reductase inhibitors (e.g., lovastatin, cerivastatin, pravastatin, atorvastatin, simvastatin, and fluvastatin), nicotinic acid, fibric acid derivatives (e.g., clofibrate, gemfibrozil, fenofibrate, bezafibrate, and ciprofibrate), probucol, neomycin, dextrothyroxine, plant-stanol esters, cholesterol absorption inhibitors (e.g., ezetimibe), implitapide, inhibitors of bile acid transporters (apical sodium-dependent bile acid transporters), regulators of hepatic CYP7a, estrogen replacement therapeutics (e.g., tamoxifen), and anti-inflammatories (e.g., glucocorticoids).

25. A compound as defined in any of embodiments 1-20 or a conjugate as defined in embodiment 21 for use as a medicament.

26. Use of a compound as defined in any of embodiments 1-20 or as conjugate as defined in embodiment 21 for the manufacture of a medicament for the treatment of abnormal levels of Apo-B100.

27. Use according to embodiment 26, wherein said abnormal levels of Apo-B100 is in the form of atherosclerosis, hypercholesterolemia or hyperlipidemia.

The invention is further illustrated in a non-limiting manner by the following examples.

### EXAMPLES

### Example 1

### Monomer Synthesis

The LNA monomer building blocks and derivatives thereof were prepared following published procedures and references cited therein, see:
WO 03/095467 A1
D. S. Pedersen, C. Rosenbohm, T. Koch (2002) Preparation of LNA Phosphoramidites, Synthesis 6, 802-808.
M. D. Sørensen, L. Kvaernø, T. Bryld, A. E. H\dot{a}kansson, B. Verbeure, G. Gaubert, P. Herdewijn, J. Wengel (2002) α-L-ribo-configured Locked Nucleic Acid (α-l-LNA): Synthesis and Properties, J. Am. Chem. Soc., 124, 2164-2176.
S. K. Singh, R. Kumar, J. Wengel (1998) Synthesis of Novel Bicyclo[2.2.1] Ribonucleosides: 2′-Amino- and 2′-Thio-LNA Monomeric Nucleosides, J. Org. Chem. 1998, 63, 6078-6079.
C. Rosenbohm, S. M. Christensen, M. D. Sørensen, D. S. Pedersen, L. E. Larsen, J. Wengel, T. Koch (2003) Synthesis of 2′-amino-LNA: a new strategy, Org. Biomol. Chem. 1, 655-663.
D. S. Pedersen, T. Koch (2003) Analogues of LNA (Locked Nucleic Acid). Synthesis of the 2′-Thio-LNA Thymine and 5-Methyl Cytosine Phosphoramidites, Synthesis 4, 578-582.

### Example 2

### Oligonucleotide Synthesis

Oligonucleotides were synthesized using the phosphoramidite approach on an Expedite 8900/MOSS synthesizer (Multiple Oligonucleotide Synthesis System) at 1 μmol or 15 μmol scale. For larger scale synthesis an Äkta Oligo Pilot was used. At the end of the synthesis (DMT-on), the oligonucleotides were cleaved from the solid support using aqueous ammonia for 1-2 h at room temperature, and further deprotected for 4 h at 65° C. The oligonucleotides were purified by reverse phase HPLC(RP-HPLC). After the removal of the DMT-group, the oligonucleotides were characterized by AE-HPLC, RP-HPLC, and CGE and the molecular mass was further confirmed by ESI-MS. See below for more details.

Preparation of the LNA-Solid Support:

Preparation of the LNA Succinyl Hemiester

5′-O-Dmt-3′-hydroxy-LNA monomer (500 mg), succinic anhydride (1.2 eq.) and DMAP (1.2 eq.) were dissolved in DCM (35 mL). The reaction was stirred at room temperature overnight. After extractions with NaH₂PO₄ 0.1 M pH 5.5 (2×) and brine (1×), the organic layer was further dried with anhydrous Na₂SO₄ filtered and evaporated. The hemiester derivative was obtained in 95% yield and was used without any further purification.

Preparation of the LNA-Support

The above prepared hemiester derivative (90 μmol) was dissolved in a minimum amount of DMF, DIEA and pyBOP (90 μmol) were added and mixed together for 1 min. This pre-activated mixture was combined with LCAA-CPG (500 Å, 80-120 mesh size, 300 mg) in a manual synthesizer and stirred. After 1.5 h at room temperature, the support was filtered off and washed with DMF, DCM and MeOH. After drying, the loading was determined to be 57 μmol/g (see Tom Brown, Dorcas J. S. Brown. Modern machine-aided methods of oligodeoxyribonucleotide synthesis. In: F. Eckstein, editor. Oligonucleotides and Analogues A Practical Approach. Oxford: IRL Press, 1991: 13-14).

Elongation of the Oligonucleotide

The coupling of phosphoramidites (A(bz), G(ibu), 5-methyl-C(bz)) or T-β-cyanoethyl-phosphoramidite) is performed by using a solution of 0.1 M of the 5′-O-DMT-protected amidite in acetonitrile and DCI (4,5-dicyanoimidazole) in acetonitrile (0.25 M) as activator. The thiolation is carried out by using xanthane chloride (0.01 M in acetonitrile:pyridine 10%). The rest of the reagents are the ones typically used for oligonucleotide synthesis. The protocol provided by the supplier was conveniently optimised.

Purification by RP-HPLC:

| | |
|---|---|
| Column: | Xterra RP₁₈ |
| Flow rate: | 3 mL/min |
| Buffers: | 0.1 M ammonium acetate pH 8 and acetonitrile |

Abbreviations

DMT: Dimethoxytrityl

DCI: 4,5-Dicyanoimidazole

DMAP: 4-Dimethylaminopyridine

DCM: Dichloromethane

DMF: Dimethylformamide

THF: Tetrahydrofurane

DIEA: N,N-diisopropylethylamine

PyBOP: Benzotriazole-1-yl-oxy-tris-pyrrolidino-phosphonium hexafluorophosphate

Bz: Benzoyl

Ibu: Isobutyryl

### Example 3

### Design of the Oligonucleotide Compound

The siRNA is a 21-nucleotide sense strand (SEQ ID NO: 27) and a 23 nucleotide antisense strand (SEQ ID NO: 28)—resulting in a two-nucleotide overhang at the 3′end of the antisense stand.

ApoB-siRNA sense 5′-GUCAUCACACUGAAUACCAA*U-3′ (SEQ ID NO: 48), ApoB-1-siRNA antisense strand 5′-AUUGGUAUUCAGUGUGAUGAc*a*C-3 (SEQ ID NO: 49) and ApoB-siRNA-Chol sense strand: 5′-GUCAUCACACUGAAUACCAAU*Chol-3′ (SEQ ID NO: 50) were synthesised by RNATEC (Leuven).

In one embodiment of the invention, SEQ ID NOS: 2-26 contains at least 3 LNA nucleotides, such as 6 or 7 LNA nucleotides like in SEQ ID NOS: 29-47.

TABLE 1

Oligonucleotide compound of the invention

| | | |
|---|---|---|
| Test | | Target |
| substance | Sequence | site |

| | | | |
|---|---|---|---|
| SEQ ID NO: 2 | 5′-GGTATTCAGTGTGATG-3′ | 10169 | Antisense motif |
| SEQ ID NO: 3 | 5′-ATTGGTATTCAGTGTG-3′ | 10172 | Antisense motif |
| SEQ ID NO: 4 | 5′-TTGTTCTGAATGTCCA-3′ | 3409 | Antisense motif |
| SEQ ID NO: 5 | 5′-TCTTGTTCTGAATGTC-3′ | 3411 | Antisense motif |
| SEQ ID NO: 6 | 5′-TGGTATTCAGTGTGAT-3′ | | Antisense motif |
| SEQ ID NO: 7 | 5′-TTGGTATTCAGTGTGA-3′ | | Antisense motif |
| SEQ ID NO: 8 | 5′-CATTGGTATTCAGTGT-3′ | 10173 | Antisense motif |
| SEQ ID NO: 9 | 5′-GCATTGGTATTCAGTG-3′ | 10174 | Antisense motif |
| SEQ ID NO: 10 | 5′-AGCATTGGTATTCAGT-3′ | 10175 | Antisense motif |
| SEQ ID NO: 11 | 5′-CAGCATTGGTATTCAG-3′ | 10176 | Antisense motif |
| SEQ ID NO: 12 | 5′-TCAGCATTGGTATTCA-3′ | | Antisense motif |
| SEQ ID NO: 13 | 5′-TTCAGCATTGGTATTC-3′ | | Antisense motif |
| SEQ ID NO: 14 | 5′-GTTCAGCATTGGTATT-3′ | | Antisense motif |
| SEQ ID NO: 15 | 5′-AGTTCAGCATTGGTAT-3′ | | Antisense motif |
| SEQ ID NO: 16 | 5′-AAGTTCAGCATTGGTA-3′ | | Antisense motif |
| SEQ ID NO: 17 | 5′-AAAGTTCAGCATTGGT-3′ | | Antisense motif |
| SEQ ID NO: 18 | 5′-ATTTCCATTAAGTTCT-3′ | 10454 | Antisense motif |
| SEQ ID NO: 19 | 5′-GGTATTTCCATTAAGT-3′ | 10457 | Antisense motif |
| SEQ ID NO: 20 | 5′-GACTCAATGGAAAAGT-3′ | 10594 | Antisense motif |
| SEQ ID NO: 21 | 5′-ATGACTCAATGGAAAA-3′ | 10596 | Antisense motif |
| SEQ ID NO: 22 | 5′-GCTAACACTAAGAACC-3′ | 10998 | Antisense motif |
| SEQ ID NO: 23 | 5′-CACTAAGAACCAGAAG-3′ | 11003 | Antisense motif |
| SEQ ID NO: 24 | 5′-CTAAGAACCAGAAGAT-3′ | 11005 | Antisense motif |
| SEQ ID NO: 25 | 5′-TGAATCGGGTCGCATC-3′ | 252 | Antisense motif |
| SEQ ID NO: 26 | 5′-TGAATCGGGTCGCATT-3′ | 252 | Antisense motif |
| SEQ ID NO: 27 | 5′-GUCAUCACACUGAAUACCAAU-3′ | | siRNA |
| SEQ ID NO: 28 | 5′-AUUGGUAUUCAGUGUGAUGACAC-3′ | | |
| SEQ ID NO: 29 | 5′- [symbol] aₛtₛtₛcₛaₛgₛtₛgₛtₛ [symbol] g-3′ | | Motif #2 |
| SEQ ID NO: 30 | 5′- [symbol] gₛgₛtₛaₛtₛtₛcₛaₛgₛ [symbol] g-3′ | | Motif #3 |
| SEQ ID NO: 31 | 5′- [symbol] gₛtₛaₛtₛtₛcₛaₛgₛ [symbol] g-3′ | | Motif #3 |
| SEQ ID NO: 32 | 5′- [symbol] tₛcₛtₛgₛaₛaₛtₛgₛ [symbol] a-3′ | | Motif #4 |
| SEQ ID NO: 33 | 5′- [symbol] gₛtₛtₛcₛtₛgₛaₛaₛ [symbol] c-3′ | | Motif #5 |
| SEQ ID NO: 34 | 5′- [symbol] gₛgₛtₛaₛtₛtₛcₛaₛ [symbol] t-3′ | | Motif #8 |
| SEQ ID NO: 35 | 5′- [symbol] tₛgₛgₛtₛaₛtₛtₛcₛ [symbol] g-3′ | | Motif #9 |
| SEQ ID NO: 36 | 5′- [symbol] tₛtₛgₛgₛtₛaₛtₛtₛ [symbol] t-3′ | | Motif #10 |
| SEQ ID NO: 37 | 5′- [symbol] cₛaₛtₛtₛgₛgₛtₛaₛtₛ [symbol] g-3′ | | Motif #11 |
| SEQ ID NO: 38 | 5′- [symbol] aₛtₛtₛgₛgₛtₛaₛtₛ [symbol] g-3′ | | Motif #11 |
| SEQ ID NO: 39 | 5′- [symbol] cₛcₛaₛtₛtₛaₛaₛgₛ [symbol] t-3′ | | Motif #19 |
| SEQ ID NO: 40 | 5′- [symbol] tₛtₛtₛcₛcₛaₛtₛtₛ [symbol] t-3′ | | Motif #19 |
| SEQ ID NO: 41 | 5′- [symbol] cₛaₛaₛtₛgₛgₛaₛaₛ [symbol] t-3′ | | Motif #20 |
| SEQ ID NO: 42 | 5′- [symbol] cₛtₛcₛaₛaₛtₛgₛg [symbol] a-3′ | | Motif #21 |
| SEQ ID NO: 43 | 5′- [symbol] aₛcₛaₛcₛtₛaₛaₛgₛ [symbol] c-3′ | | Motif #22 |
| SEQ ID NO: 44 | 5′- [symbol] aₛaₛgₛaₛaₛcₛcₛaₛ [symbol] g-3′ | | Motif #23 |
| SEQ ID NO: 45 | 5′- [symbol] gₛaₛaₛcₛcₛaₛgₛaₛ [symbol] t-3′ | | Motif #24 |
| SEQ ID NO: 46 | 5′- [symbol] tₛcₛgₛgₛgₛtₛcₛgₛ [symbol] c-3′ | | Motif #25 |
| SEQ ID NO: 47 | 5′- [symbol] tₛcₛgₛgₛgₛtₛcₛgₛ [symbol] t-3′ | | Motif #26 |
| SEQ ID NO: 48 | 5′-GUCAUCACACUGAAUACCAAₛU-3′ (290.3) | | Unconjugated |
| SEQ ID NO: 49 | 5′-AUUGGUAUUCAGUGUGAUGA [symbol] C-3′ (290.5) | | siRNA |
| ApoB-si RNA | | | |
| (RNA-TEC290.3 | | | |
| RNA-TEC290.5) | | | |
| SEQ ID NO: 50 | 5′-GUCAUCACACUGAAUACCAAUₛ-Chol-3′ (290.4) | | Cholesteryl |
| ApoB-siRNA- | | | conjugated |
| Chol | | | siRNA |
| (SEQ ID NO: 51 | 5′-AUUGGUAUUCAGUGUGAUGA [symbol] C-3′ (290.5) | | |
| RNA-TEC290.4 | | | |
| RNA-TEC290.5) | | | |

In SEQ ID NOS: 27, 28, 48, 49, 50, upper case letters indicates ribonucleotide units and subscript “s” represents phosphorothiote linkage.

SEQ ID No 30 is an interesting compound according to the invention.

### Example 4

### Stability of LNA Compounds in Human or Rat Plasma

LNA oligonucleotide stability was tested in plasma from humans or rats (it could also be mouse, monkey or dog plasma). In 45 μl plasma 5 μl oligonucleotide is added (a final concentration of 20 μM). The oligos are incubated in plasma for times ranging from 0 h-96 h at 37° C. (the plasma is tested for nuclease activity up to 96 h and shows no difference in nuclease cleavage-pattern). At the indicated time the sample were snap-frozen in liquid nitrogen. 2 μl (equals 40 μmol) oligonucleotide in plasma was diluted by adding 15 μl of water and 3 μl 6× loading dye (Invitrogen). As marker a 10 bp ladder (Invitrogen 10821-015) is used. To 1 μl ladder 1 μl 6× loading and 4 μl water was added. The samples were mixed, heated to 65° C. for 10 min and loaded to a prerun gel (16% acrylamide, 7 M UREA, 1×TBE, pre-run at 50 Watt for 1 h) and run at 50-60 Watt for 2½ h. Subsequently the gel was stained with 1× SyBR gold (molecular probes) in 1×TBE for 15 min. The bands were visualised using a phosphorimager from Biorad.

### Example 5

### In Vitro Model: Cell Culture

The effect of antisense compounds on target nucleic acid expression can be tested in any of a variety of cell types provided that the target nucleic acid is present at measurable levels. Target can be expressed endogenously or by transient or stable transfection of a nucleic acid encoding said nucleic acid.

The expression level of target nucleic acid can be routinely determined using, for example, Northern blot analysis, Quantitative PCR, Ribonuclease protection assays. The following cell types are provided for illustrative purposes, but other cell types can be routinely used, provided that the target is expressed in the cell type chosen.

Cells were cultured in the appropriate medium as described below and maintained at 37° C. at 95-98% humidity and 5% CO₂. Cells were routinely passaged 2-3 times weekly.

BNCL-2: Mouse liver cell line BNCL-2 was purchased from ATCC and cultured in DMEM (Sigma) with 10% FBS+Glutamax I+non-essential amino acids+gentamicin.

Hepa1-6: Mouse liver cell line Hepa1-6 was purchased from ATCC and cultured in DMEM (Sigma) with 10% FBS+Glutamax I+non-essential amino acids+gentamicin.

HepG2: Human liver cell line HepG2 was purchased from ATCC and cultured in Eagle MEM (Sigma) with 10% FBS+Glutamax I+non-essential amino acids+gentamicin.

### Example 6

### In Vitro Model: Treatment with Antisense Oligonucleotide

Cell culturing and transfections: BNCL-2 or Hepa1-6 cells were seeded in 12-well plates at 37° C. (5% CO₂) in growth media supplemented with 10% FBS, Glutamax I and Gentamicin. When the cells were 60-70% confluent, they were transfected in duplicates with different concentrations of oligonucleotides (0.04-25 nM) using Lipofectamine 2000 (5 μg/mL). Transfections were carried out essentially as described by Dean et al. (1994, JBC 269:16416-16424). In short, cells were incubated for 10 min. with Lipofectamine in OptiMEM followed by addition of oligonucleotide to a total volume of 0.5 mL transfection mix per well. After 4 hours, the transfection mix was removed, cells were washed and grown at 37° C. for approximately 20 hours (mRNA analysis and protein analysis in the appropriate growth medium. Cells were then harvested for protein and RNA analysis.

### Example 7

### In Vitro Model: Extraction of RNA and cDNA Synthesis

Total RNA Isolation

Total RNA was isolated using RNeasy mini kit (Qiagen). Cells were washed with PBS, and Cell Lysis Buffer (RTL, Qiagen) supplemented with 1% mercaptoethanol was added directly to the wells. After a few minutes, the samples were processed according to manufacturer's instructions.

First Strand Synthesis

First strand synthesis was performed using either OmniScript Reverse Transcriptase kit or M-MLV Reverse transcriptase (essentially as described by manufacturer (Ambion)) according to the manufacturer's instructions (Qiagen). When using OmniScript Reverse Transcriptase 0.5 μg total RNA each sample, was adjusted to 12 μl and mixed with 0.2 μl poly (dT)₁₂₋₁₈ (0.5 μg/μl) (Life Technologies), 2 μl dNTP mix (5 mM each), 2 μl 10×RT buffer, 0.5 μl RNAguard™ RNase Inhibitor (33 units/mL, Amersham) and 1 μl OmniScript Reverse Transcriptase followed by incubation at 37° C. for 60 min. and heat inactivation at 93° C. for 5 min.

When first strand synthesis was performed using random decamers and M-MLV-Reverse Transcriptase (essentially as described by manufacturer (Ambion)) 0.25 μg total RNA of each sample was adjusted to 10.8 μl in H₂O. 2 μl decamers and 2 μl dNTP mix (2.5 mM each) was added. Samples were heated to 70° C. for 3 min. and cooled immediately in ice water and added 3.25 μl of a mix containing (2 μl 10×RT buffer; 1 μl M-MLV Reverse Transcriptase; 0.25 μl RNAase inhibitor). cDNA is synthesized at 42° C. for 60 min followed by heating inactivation step at 95° C. for 10 min and finally cooled to 4° C.

### Example 8

### In Vitro and In Vivo Model: Analysis of Oligonucleotide Inhibition of Apo-B100 Expression by Real-Time PCR

Antisense modulation of Apo-B100 expression can be assayed in a variety of ways known in the art. For example, Apo-B100 mRNA levels can be quantitated by, e.g., Northern blot analysis, competitive polymerase chain reaction (PCR), or real-time PCR. Real-time quantitative PCR is presently preferred. RNA analysis can be performed on total cellular RNA or mRNA.

Methods of RNA isolation and RNA analysis such as Northern blot analysis is routine in the art and is taught in, for example, Current Protocols in Molecular Biology, John Wiley and Sons.

Real-time quantitative (PCR) can be conveniently accomplished using the commercially iQ Multi-Color Real Time PCR Detection System available from BioRAD. Real-time Quantitative PCR is a technique well known in the art and is taught in for example Heid et al. Real time quantitative PCR, Genome Research (1996), 6: 986-994.

Real-Time Quantitative PCR Analysis of Apo-B100 mRNA Levels

To determine the relative mouse ApoB mRNA level in treated and untreated samples, the generated cDNA was used in quantitative PCR analysis using an iCycler from BioRad.

To 8 μl of 5-fold (Gapdh and Beta-actin) diluted cDNA was added 52 μl of a mix containing 29.5 μl Platinum qPCR

Supermix-UDG (in-vitrogen), 1030 nM of each primer, 0.57×SYBR Green (Molecular probes) and 11.4 nM Fluorescein (Molecular probes).

Duplicates of 25 μl was used for Q-PCR: 50° C. for 120 sec., 95° C. for 120 sec. and 40 cycles [95° C. for 30 sec. and 60° C. for 60 sec.].

ApoB expression was quantified using a 50-fold diluted cDNA and a standard Q-PCR protocol. The primers (final conc of respectively forward and reverse primers 0.6 μM and 0.9 μM) and probe (final conc. 0.1 μM) were mixed with 2× Platinum Quantitative PCR SuperMix UDG (cat. # 11730, Invitrogen) and added to 3.3 μl cDNA to a final volume of 25 μl. Each sample was analysed in duplicates. PCR program: 50° C. for 2 minutes, 95° C. for 10 minutes followed by 40 cycles of 95° C., 15 seconds, 60° C., 1 minutes.

ApoB mRNA expression was normalized to mouse β-actin or Gapdh mRNA which was similarly quantified using Q-PCR.

Primers:

| | |
|---|---|
| mGapdh: | |
| 5′-agcctcgtcccgtagacaaaat-3′ | (SEQ ID NO: 51) |
| and | |
| 5′-gttgatggcaacaatctccacttt-3′ | (SEQ ID NO: 52) |
| mβ-actin: | |
| 5′-ccttccttcttgggtatggaa-3′ | (SEQ ID NO: 53) |
| and | |
| 5′-gctcaggaggagcaatgatct-3′ | (SEQ ID NO: 54) |
| mApoB: | |
| 5′-gcccattgtggacaagttgatc-3′ | (SEQ ID NO: 55) |
| and | |
| 5′-ccaggacttggaggtcttgga-3′ | (SEQ ID NO: 56) |
| mApoB Taqman probe: | |
| 5′-fam-aagccagggcctatctccgcatcc-3′ | (SEQ ID NO: 57) |

2-fold dilutions of cDNA synthesised from untreated mouse Hepatocyte cell line (Hepa1-6 cells) (diluted 5 fold and expressing both ApoB and β-actin or Gapdh) was used to prepare standard curves for the assays. Relative quantities of ApoB mRNA were determined from the calculated Threshold cycle using the iCycler iQ Real Time Detection System software.

### Example 9

### In Vitro Analysis: Western Blot Analysis of Apo-B100 Protein Levels

The in vitro effect of Apo-B100 oligoes on Apo-B100 protein levels in transfected cells was determined by Western Blotting.

Cells were harvested and lysed in 50 mM Tris-HCl pH 6.8, 10% glycerol, 2.5% SDS, 5 mM DTT and 6 M urea supplemented with protease inhibitor cocktail (Roche). Total protein concentrations were measured using a BCA protein assay kit (Pierce). 50 μg total protein was run on 10-12% Bis-Tris gels in MOPS buffer or on 3-8% Tris Acetate gels and blotted onto a PVDF membranes according to manufacture's instructions (Invitrogen). After overnight incubation in blocking buffer (PBS-T supplemented with 5% low fat milk powder), the membranes were incubated overnight with primary antibody detecting ApoB-100. As control of loading, tubulin or actin were detected using monoclonal antibodies from Neomarker. Membranes were then incubated with secondary antibodies and ApoB-100 was visualized using a chromogenic immunodetection kit (Invitrogen) or a chemiluminescens ECL⁺ detection kit (Amersham).

### Example 10

### In Vitro Analysis: Antisense Inhibition of Human Apo-B100 Expression Using Antisense Oligonucleotides

In accordance with the present invention, a series of oligonucleotides were designed to target different regions of the human Apo-B100 RNA. See Table 1 Oligonucleotide compounds were evaluated for their potential to knockdown Apo-B100 mRNA in mouse hepatocytes (Hepa1-6 cells) following lipid-assisted uptake of SEQ ID NO: 29, siRNA (unmodified) or cholesteryl modified siRNA (FIG. 1A) and comparison of knockdown of ApoB-100 in BNLCL2 by the two LNA oligonucleotides SEQ ID No:29 and SEQ ID No:30 (FIG. 1B) and in Hepa 1-6 cells by SEQ ID No:29 and SEQ ID No:37 (FIG. 4).

The data are presented as percentage downregulation relative to mock transfected cells. Transcript steady state was monitored by Real-time PCR and normalised to the GAPDH transcript steady state.

### Example 11

### In-Vivo Target Downregulation of LNA Containing Oligonucleotide Compounds

C57BL/6 mice (20 g) received 6.25, 12.5, 25 or 50 mg/kg i.v. on three consecutive days (group size of 7 mice). We have dosed with less antisense oligonucleotide since the molecular weight of a siRNA-Chol compared to an antisense oligonucleotide is approximately 3:1. All siRNA's and antisense oligonucleotides were dissolved in 0.9% saline (NaCl) and given at 10 mL/kg body weight (˜0.2 ml per injection). At sacrifice the weight of the liver was recorded. Tissues for measurement of ApoB mRNA expression was stored in RNA later (Ambion) at −20° C. until use. mRNA analysis on Jejunum and Liver and total cholesterol in plasma were performed 24 h after last i.v. injection. (see FIGS. 2A and 2B)

### Example 12

### Cholesterol Levels in Plasma

Total cholesterol level was measured in plasma using a colometric assay Cholesterol CP from ABX Pentra. The cholesterol is measured following enzymatic hydrolysis and oxidation. 21.5 μL water was added to 1.5 μL plasma. 250 μL reagent is added and within 5 min the cholesterol content is measured at a wavelength of 540 nM. Measurements on each animal was made in duplicates. The sensitivity and linearity was tested with 2 fold diluted control compound (ABX Pentra N control). The relative Cholesterol level was determined by subtraction of the background and presented relative to the cholesterol levels in plasma of saline treated mice. (see FIG. 3)

### Example 13

### In-Vivo Target Down-Regulation of LNA Oligonucleotide Compounds

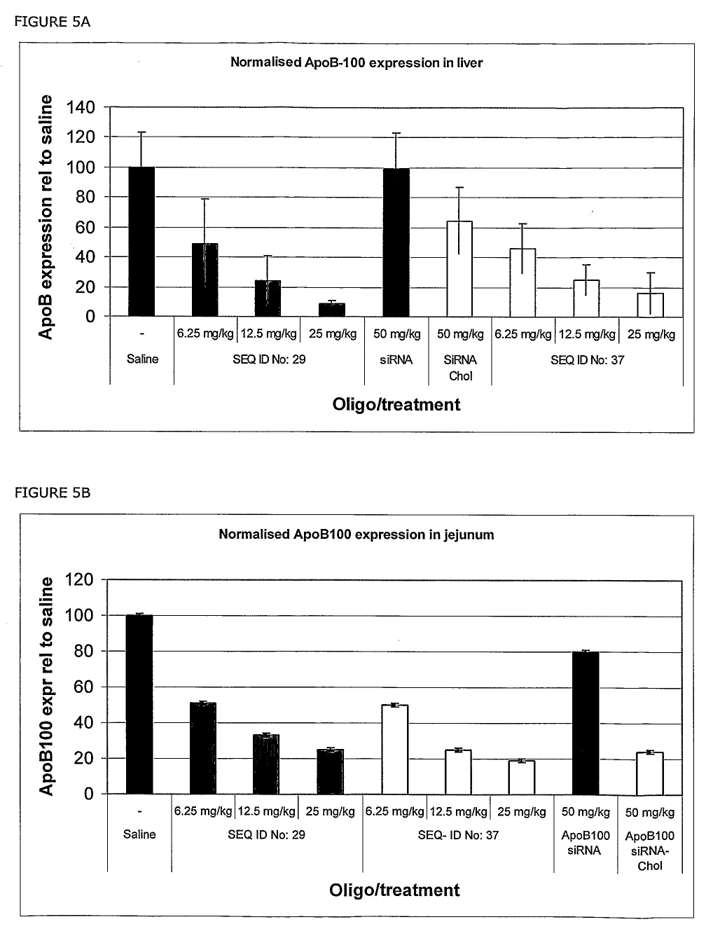

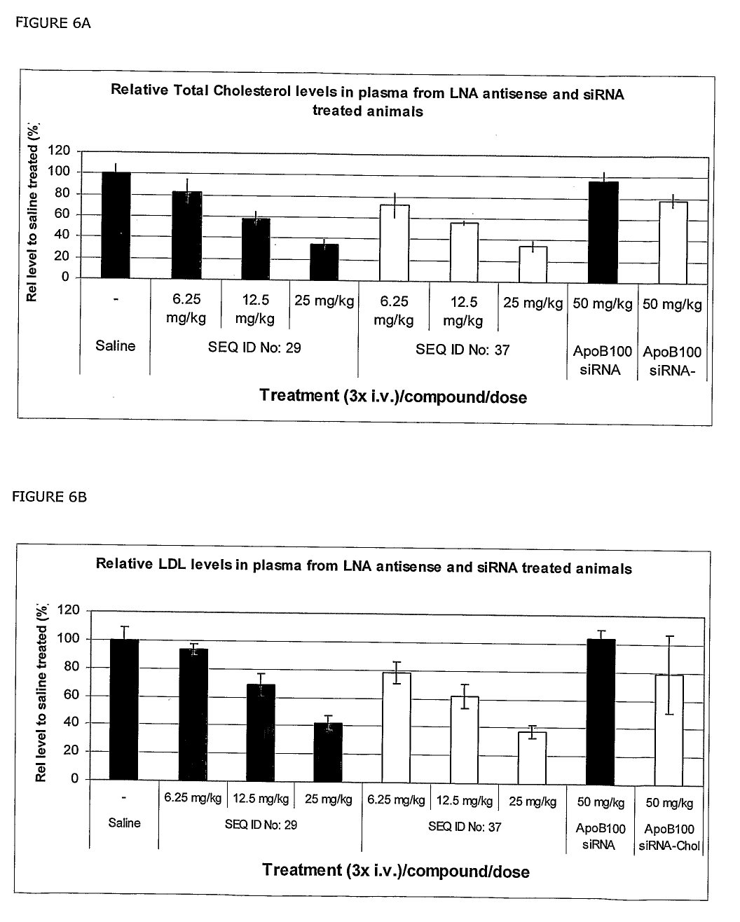

C57BL/6 mice (20 g) received 6.25, 12, or, 25 mg/kg i.v. on three consecutive days (group size of 7 mice). The antisense oligonucleotides (SEQ ID NO: 29 and SEQ ID NO: 37) were dissolved in 0.9% saline (NaCl) and given at 10 mL/kg body weight (˜0.2 mL per injection). Tissues for measurement of ApoB mRNA expression was stored in RNA later (Ambion) at −20° C. until use. mRNA analysis on Jejunum and Liver, total- and LDL cholesterol in plasma were performed 24 h after last i.v. injection. (see FIGS. 5A, 5B, 6A and 6B).

### Example 14

### Oral Administration of LNA Oligonucleotide Compounds to Mice

C57BL/6 mice (20 g) received 10 mL/kg, i.e. 0.2 mL, a freshly prepared formulation of 1.0 mL oligonucleotide (SEQ ID NO: 29 OR SEQ ID NO: 37) in sterile H₂O (7.5 mg/ml), 0.1 mL Tween80, 1.9 mL olive oil. Final concentration of oligonucleotide compound: 2.5 mg/mL. The formulation was shaken for 1 min; ultra sound sonicated for 5 min (repeated 3 times). No negative effects were observed.

### Example 15

### In Vitro Analysis: Dose Response in Cell Culture (Human Hepotocyte Huh-7)/Antisense Inhibition of Human Apo-B100 Expression

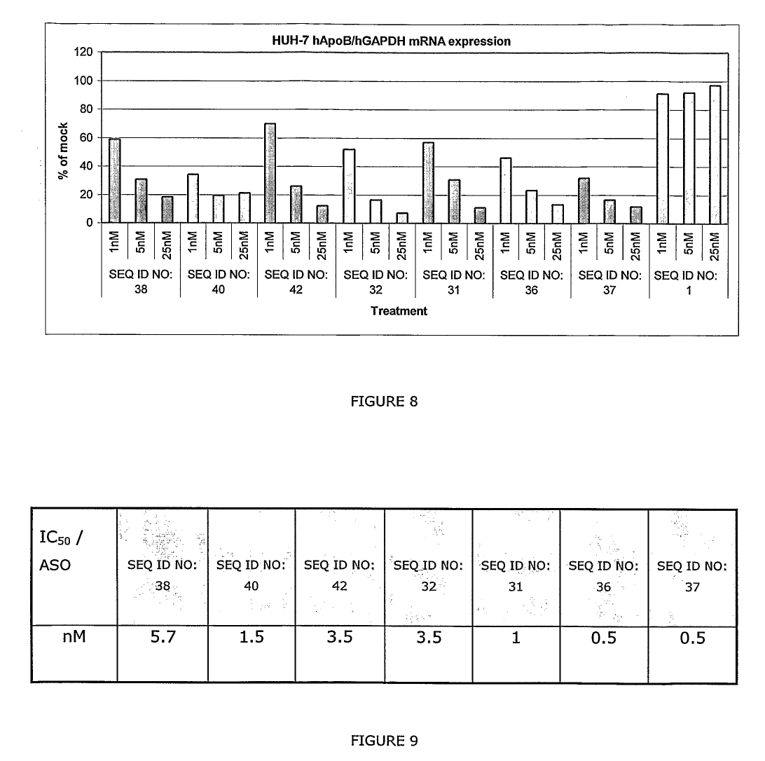

In accordance with the present invention, a series of oligonucleotides were designed to target different regions of the human Apo-B100 mRNA. See Table 1 Oligonucleotide compounds were evaluated for their potential to knockdown Apo-B100 mRNA in Human hepatocytes (Huh-7 cells) following lipid-assisted uptake of SEQ ID NO: 31-32, 36-38 and 40-42 (FIG. 8). The experiment was performed as described in examples 5-8. The results showed very potent down regulation (>80%) with 25 nM for all compounds. However at 1 nM only 2 compounds resulted in a ApoB-100 mRNA down regulation as high as 700% (SEQ ID NO: 37 and 40, which is a very potent down regulation (FIG. 8).

### Example 16

### IC₅₀ for 7 selected LNA Antisense Oligonucleotides in Cell Culture (Human Hepatocyte Huh-7)

The 7 antisense oligonucleotides with the best in vitro down regulation was selected for an IC₅₀ study to determine the concentration of the antisense oligonucleotide to give a 50% inhibition of ApoB-100 mRNA expression. The experiment was made as described in examples 5-8. Only SEQ ID NO: 36 and 37 had an IC50 of about 1 nM, whereas SEQ ID NO:38 had IC50 as high as 5.7 (FIG. 9). An IC50 of 0.5 nM indicates a very poteny compound, which for SEQ ID NO: 37 has been confirmed by in vivo data (examples 17 and 18).

### Example 17

### Duration of Action of Dosing SEQ ID NO 37 Once, Twice or Three Times

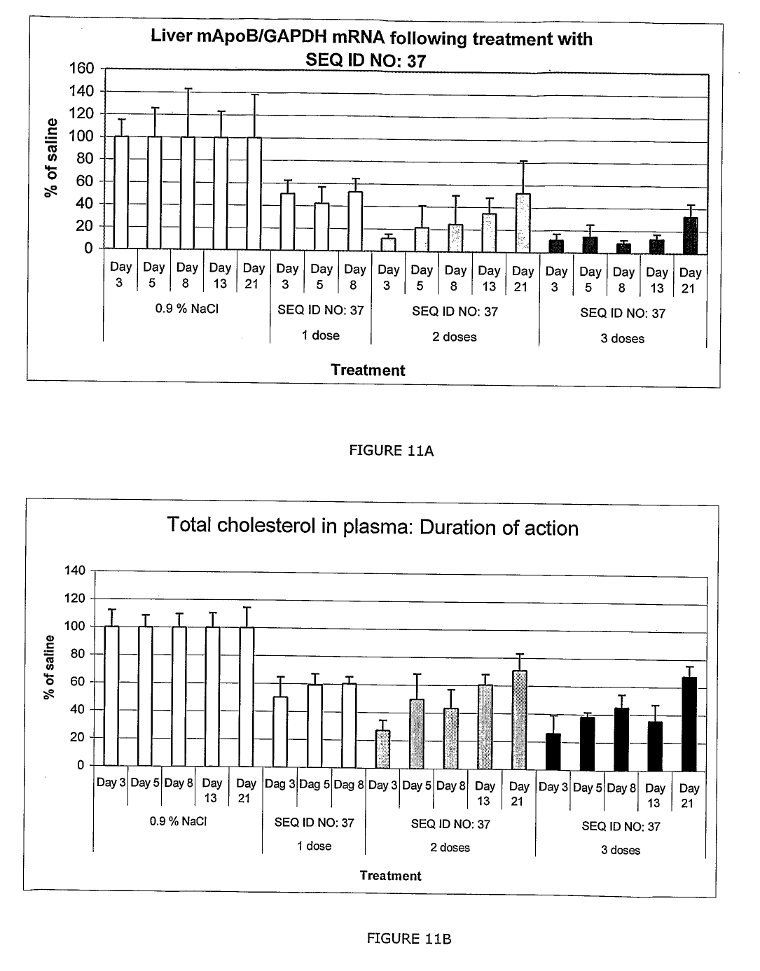

C57BL/6 mice (20 g) received 6.25 or 25 mg/kg/dose i.p. on one, two or three consecutive days (group size of 5 mice). All antisense oligonucleotides were dissolved in 0.9% saline (NaCl) and administered at 10 mL/kg body weight (˜0.2 ml per injection). At sacrifice (days 3, 5, 8, 13 and 21) the weight of the liver was recorded. Tissues for measurement of ApoB mRNA expression were stored in RNA later (Ambion) at −20° C. until use. mRNA analysis on Liver was performed at sacrifice whereas LDL- and total cholesterol in plasma were performed 24 h, 2 or 3 and, 6, 11, and 19 days after last l.9. injection. (see FIG. 11). This study showed a very potent down regulation of ApoB-100 mRNA following dosing SEQ ID NO: 37: One dose resulted in af ApoB mRNA expression of 45-60% from day 3 to day 8 after dosing, whereas 3 doses resulted in 85-90% down regulation at day 13 and about 70% at day 21, showing a duration of action longer than 20 days in liver when 2 or 3 doses were administered. ApoB-100 mRNA expression and total cholesterol were measured as described in examples 8 and 12.

### Example 18

### Dose Regimes of SEQ ID NO 37

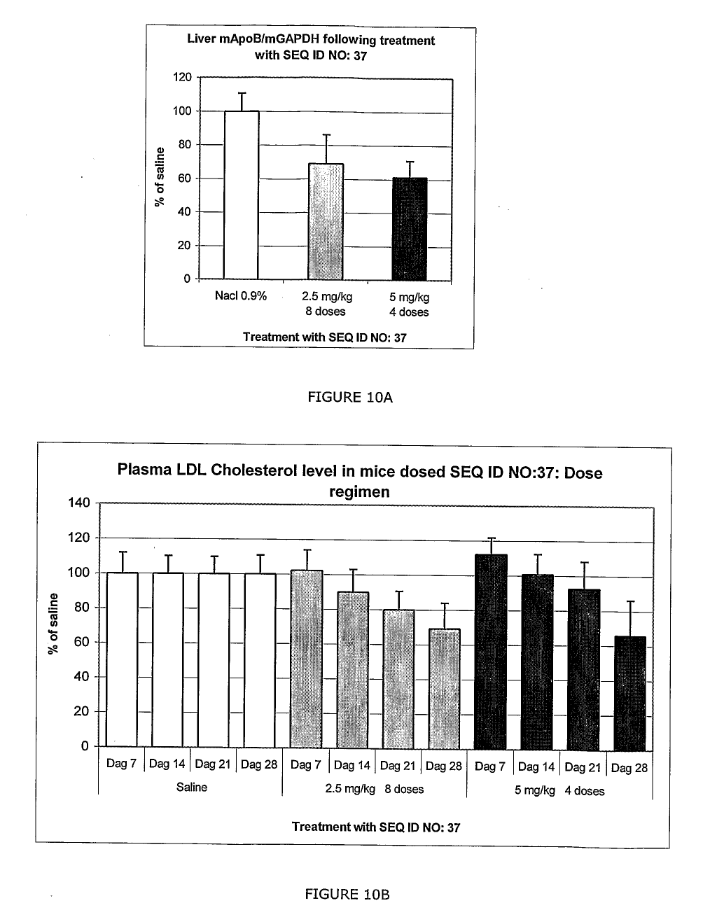

C57BL/6 mice (20 g) received 2. mg/kg/dose i.p. twice weekly for 4 weeks or 5 mg/kg/dose once weekly for 4 weeks (group size of 5 mice) to examine the effect on target (ApoB-100) mRNA down-regulation and on plasma cholesterol level (collected once weekly). The antisense oligonucleotide was dissolved in 0.9% saline (NaCl) and administered at 10 mL/kg body weight (˜0.2 ml per injection). At sacrifice (day 28) the weight of the liver was recorded. Tissues for measurement of ApoB mRNA expression were stored in RNA later (Ambion) at −20° C. until use. mRNA analysis on Liver was performed at sacrificed whereas LDL cholesterol level in plasma were determined days 7, 14, 21 and 28 (see FIG. 10). The results showed a linear decrease in LDL cholesterol level over time resulting in a 300% reduction at day 28 compared to day 7 and the saline group after dosing 2.5 mg/kg/dose twice weekly. Similar results were obtained dosing the same total amount of antisense oligonucleotide but dosing 5 mg/kg/dose only once weekly. Furthermore, the ApoB-100 mRNA level in liver at sacrifice (day 28) showed a down regulation of 30-40% after dosing 20 mg/kg over 28 days independent of the dose regimen (one or two doses weekly). These results show a significant down regulation of ApoB-100 mRNA even at low doses of SEQ ID NO 37, and that this down regulation has an impact on the therapeutic read out measured as a 30% reduction in plasma LDL-cholesterol. ApoB-100 mRNA expression and Cholesterol levels were measured as described in examples 8 and 12.

1-35. (canceled)

36. An oligomeric compound consisting of a total of 12-50 nucleotides and/or nucleotide analogues, wherein the compound comprises a subsequence of at least 10 nucleotides or nucleotide analogues, the subsequence corresponding to a sequence selected from the group consisting of SEQ ID NOS: 59, 60, 61, 62, 63, 2, 3, 6, 7, 8, 9, 10, 11, 12, 13, 14, 15, 16 and 17, wherein the compound comprises at least 3 nucleotide analogues, and wherein the sub-sequence may comprise 1 or 2 mismatches when compared to the corresponding sequence in the sequence selected from the group consisting of SEQ ID NOS: 59, 60, 61, 62, 63, 2, 3, 6, 7, 8, 9, 10, 11, 12, 13, 14, 15, 16 and 17.

37. The compound according to claim 36, wherein the sub-sequence comprises the at least three nucleotide analogues.

38. The compound according to claim 36, wherein the subsequence comprises a stretch of 2-6 nucleotide analogues, followed by a stretch of 4-12 nucleotides, which is followed by a stretch of 2-6 nucleotide analogues.

39. The compound according to claim 38 consisting of from 12-25 nucleotides or nucleotide analogues.

40. The compound according to claim 39, wherein at least one of the nucleotide analogues is a locked nucleic acid (LNA), such as at least three, or all nucleotide analogues are LNA.

41. The compound according to claim 36, which has the formula
5′-[(LNA)₂₋₆-(DNA/RNA)₄₋₁₂-(LNA)₂₋₆(DNA/RNA)₀₋₁]-
3′
or
5′-[(LNA)₃₋₄-(DNA/RNA)₈₋₉-(LNA)₃(DNA/RNA)₁]-3′
wherein “LNA” designates an LNA nucleotide and “DNA” and “RNA” designate a deoxyribonucleotide and a ribonucleotide, respectively.

42. The compound according to claim 41 consisting of 15, 16, 17, 18, 19, 20, 21, or 22 nucleotides or nucleotide analogues.

43. The compound according to claim 41 consisting of 15 or 16 nucleotides or nucleotide analogues.

44. The compound according to claim 41, wherein the nucleotides comprise a linkage group selected from the group consisting of a phosphate group, a phosphorothioate group and a boranophosphate group, the internucleoside linkage may be —O—P(O)₂—O—, —O—P(O,S)—O.

45. The compound according to claim 44, wherein the linkage is a phosphorothioate group.

46. The compound according to claim 44, wherein all linkages are phosphorothioate groups.

47. The compound according to claim 41, which comprises from 4-12 nucleotide analogues.

48. The compound according to claim 41 which comprises 6 or 7 nucleotide analogues.

49. The compound according to claim 41, wherein LNA is selected from beta-D-oxy-LNA, alpha-L-oxy-LNA, beta-D-amino-LNA and beta-D-thio-LNA.

50. The compound according to claim 41, wherein the subsequence is selected form the group consisting of: SEQ ID NO:63, SEQ ID NO:2, SEQ ID NO:3, SEQ ID NO:9, SEQ ID NO:10, SEQ ID NO:11, and SEQ ID NO:12.

51. The compound according to claim 41, wherein the subsequence is selected form the group consisting of: SEQ ID NO:9, SEQ ID NO:11, and SEQ ID NO:12.

52. The compound according to claim 41 which consist of 13 or 14 nucleotides or nucleotide analogues.

53. The compound according to claim 51 which consists of 13 or 14 nucleotides or nucleotide analogues.

54. A conjugate comprising the compound according to claim 41 and at least one non-nucleotide or non-polynucleotide moiety covalently attached to the compound.

55. A pharmaceutical composition comprising a compound as defined in claim 41 and a pharmaceutically acceptable diluent, carrier or adjuvant.

56. A pharmaceutical composition comprising a compound as defined in claim 53 and a pharmaceutically acceptable diluent, carrier or adjuvant.

57. A method of treating a subject suffering from a disease or condition selected from atherosclerosis, hypercholesterolemia and hyperlipidemia, the method comprising the step of administering a pharmaceutical composition according to claim 55 to the subject in need thereof.

58. A method of treating a subject suffering from a disease or condition selected from atherosclerosis, hypercholesterolemia and hyperlipidemia, the method comprising the step of administering a pharmaceutical composition according to claim 56 to the subject in need thereof.


SEQUENCE LISTING (68 sequences)

SEQ ID NO: 1 - DNA, 16 bp, artificial
  Scambled LNA oligonucloeitde control
cgtcagtatgcgaatc

SEQ ID NO: 2 - DNA, 16 bp, Artificial
  Antisense motif
ggtattcagtgtgatg

SEQ ID NO: 3 - DNA, 16 bp, Artificial
  Antisense motif
attggtattcagtgtg

SEQ ID NO: 4 - DNA, 16 bp, Artificial
  Antisense motif
ttgttctgaatgtcca

SEQ ID NO: 5 - DNA, 16 bp, Artificial
  Antisense motif
tcttgttctgaatgtc

SEQ ID NO: 6 - DNA, 16 bp, Artificial
  Antisense motif
tggtattcagtgtgat

SEQ ID NO: 7 - DNA, 16 bp, Artificial
  Antisense motif
ttggtattcagtgtga

SEQ ID NO: 8 - DNA, 16 bp, Artificial
  Antisense motif
cattggtattcagtgt

SEQ ID NO: 9 - DNA, 16 bp, Artificial
  Antisense motif
gcattggtattcagtg

SEQ ID NO: 10 - DNA, 16 bp, Artificial
  Antisense motif
agcattggtattcagt

SEQ ID NO: 11 - DNA, 16 bp, Artificial
  Antisense motif
cagcattggtattcag

SEQ ID NO: 12 - DNA, 16 bp, Artificial
  Antisense motif
tcagcattggtattca

SEQ ID NO: 13 - DNA, 16 bp, Artificial
  Antisense motif
ttcagcattggtattc

SEQ ID NO: 14 - DNA, 16 bp, Artificial
  Antisense motif
gttcagcattggtatt

SEQ ID NO: 15 - DNA, 16 bp, Artificial
  Antisense motif
agttcagcattggtat

SEQ ID NO: 16 - DNA, 16 bp, Artificial
  Antisense motif
aagttcagcattggta

SEQ ID NO: 17 - DNA, 16 bp, Artificial
  Antisense motif
aaagttcagcattggt

SEQ ID NO: 18 - DNA, 16 bp, Artificial
  Antisense motif
atttccattaagttct

SEQ ID NO: 19 - DNA, 16 bp, Artificial
  Antisense motif
ggtatttccattaagt

SEQ ID NO: 20 - DNA, 16 bp, Artificial
  Antisense motif
gactcaatggaaaagt

SEQ ID NO: 21 - DNA, 16 bp, Artificial
  Antisense motif
atgactcaatggaaaa

SEQ ID NO: 22 - DNA, 16 bp, Artificial
  Antisense motif
gctaacactaagaacc

SEQ ID NO: 23 - DNA, 16 bp, Artificial
  Antisense motif
cactaagaaccagaag

SEQ ID NO: 24 - DNA, 16 bp, Artificial
  Antisense motif
ctaagaaccagaagat

SEQ ID NO: 25 - DNA, 16 bp, Artificial
  Antisense motif
tgaatcgggtcgcatc

SEQ ID NO: 26 - DNA, 16 bp, Artificial
  Antisense motif
tgaatcgggtcgcatt

SEQ ID NO: 27 - RNA, 21 nt, Artificial
  siRNA
gucaucacacugaauaccaau

SEQ ID NO: 28 - RNA, 23 nt, Artificial
  siRNA
auugguauucagugugaugacac

SEQ ID NO: 29 - DNA, 16 bp, Artificial
  Motif #2
ggtattcagtgtgatg

SEQ ID NO: 30 - DNA, 16 bp, Artificial
  Motif #3
attggtattcagtgtg

SEQ ID NO: 31 - DNA, 16 bp, Artificial
  Motif #3
attggtattcagtgtg

SEQ ID NO: 32 - DNA, 16 bp, Artificial
  Motif #4
ttgttctgaatgtcca

SEQ ID NO: 33 - DNA, 16 bp, Artificial
  Motif #5
tcttgttctgaatgtc

SEQ ID NO: 34 - DNA, 16 bp, Artificial
  Motif #8
cattggtattcagtgt

SEQ ID NO: 35 - DNA, 16 bp, Artificial
  Motif #9
gcattggtattcagtg

SEQ ID NO: 36 - DNA, 16 bp, Artificial
  Motif #10
agcattggtattcagt

SEQ ID NO: 37 - DNA, 16 bp, Artificial
  Motif #11
cagcattggtattcag

SEQ ID NO: 38 - DNA, 16 bp, Artificial
  Motif #11
cagcattggtattcag

SEQ ID NO: 39 - DNA, 16 bp, Artificial
  Motif #19
atttccattaagttct

SEQ ID NO: 40 - DNA, 16 bp, Artificial
  Motif #19
ggtatttccattaagt

SEQ ID NO: 41 - DNA, 16 bp, Artificial
  Motif #20
gactcaatggaaaagt

SEQ ID NO: 42 - DNA, 16 bp, Artificial
  Motif #21
atgactcaatggaaaa

SEQ ID NO: 43 - DNA, 16 bp, Artificial
  Motif #22
gctaacactaagaacc

SEQ ID NO: 44 - DNA, 16 bp, Artificial
  Motif #23
cactaagaaccagaag

SEQ ID NO: 45 - DNA, 16 bp, Artificial
  Motif #24
ctaagaaccagaagat

SEQ ID NO: 46 - DNA, 16 bp, Artificial
  Motif #25
tgaatcgggtcgcatc

SEQ ID NO: 47 - DNA, 16 bp, Artificial
  Motif #26
tgaatcgggtcgcatt

SEQ ID NO: 48 - RNA, 21 nt, Artificial
  Unconjugated siRNA
gucaucacacugaauaccaau

SEQ ID NO: 49 - RNA, 23 nt, Artificial
  Unconjugated siRNA
auugguauucagugugaugacac

SEQ ID NO: 50 - RNA, 21 nt, Artificial
  Cholesterol conjugated siRNA
gucaucacacugaauaccaau

SEQ ID NO: 51 - RNA, 23 nt, Artificial
  Cholesterol conjugated siRNA
auugguauucagugugaugacac

SEQ ID NO: 52 - DNA, 22 bp, Artificial
  oligonucleotide primer
agcctcgtcccgtagacaaaat

SEQ ID NO: 53 - DNA, 24 bp, Artificial
  oligonucleotide primer
gttgatggcaacaatctccacttt

SEQ ID NO: 54 - DNA, 21 bp, Artificial
  oligonucleotide primer
ccttccttcttgggtatggaa

SEQ ID NO: 55 - DNA, 21 bp, Artificial
  oligonucleotide primer
gctcaggaggagcaatgatct

SEQ ID NO: 56 - DNA, 22 bp, Artificial
  oligonucleotide primer
gcccattgtggacaagttgatc

SEQ ID NO: 57 - DNA, 21 bp, Artificial
  oligonucleotide primer
ccaggacttggaggtcttgga

SEQ ID NO: 58 - DNA, 24 bp, Artificial
  mApoB Taqman probe
aagccagggcctatctccgcatcc

SEQ ID NO: 59 - DNA, 8 bp, Artificial
  target motif
gtattcag

SEQ ID NO: 60 - DNA, 8 bp, Artificial
  target motif
ggtattca

SEQ ID NO: 61 - DNA, 8 bp, Artificial
  target motif
tcagcatt

SEQ ID NO: 62 - DNA, 8 bp, Artificial
  target motif
gcattggt

SEQ ID NO: 63 - DNA, 29 bp, Artificial
  target motif
aaagttcagcattggtattcagtgtgatg

SEQ ID NO: 64 - DNA, 19 bp, Artificial
  target motif
ggtatttccattaagttct

SEQ ID NO: 65 - DNA, 18 bp, Artificial
  target motif
atgactcaatggaaaagt

SEQ ID NO: 66 - DNA, 24 bp, Artificial
  target motif
cactaagaaccagaaccagaagat

SEQ ID NO: 67 - DNA, 16 bp, Artificial
  target motif
tgaatcgggtcgcaty

SEQ ID NO: 68 - DNA, 18 bp, Artificial
  Subsequence/ preferred oligo motif
tcttgttctgaatgtcca